(19) United States
(12) Patent Application Publication
(10) Pub. No.: US 2008/0108632 A1
(43) Pub. Date: May 8, 2008

(54) HCV PROTEASE INHIBITORS

(75) Inventors: Chu-chung Lin, Taipei City (TW); Pei-chin Cheng, Changhua County (TW); Yo-chin Liu, Taipei County (TW); Chen-fu Liu, Taipei City (TW); Chi-Hsin Richard King, Holladay, UT (US)

Correspondence Address: OCCHIUTI ROHLICEK & TSAO, LLP, 10 FAWCETT STREET, CAMBRIDGE, MA, 02138, US

(73) Assignee: TaiGen Biotechnology Co., Ltd., Taipei (TW)

(21) Appl. No.: 11/934,151

(22) Filed: Nov. 2, 2007

Related U.S. Application Data
(60) Provisional application No. 60/856,231, filed on Nov. 2, 2006.

(51) Int. Cl. A61K 31/4015; A61K 31/427; A61K 31/497; C07D 241/12; C07D 417/14; C07D 487/04

(52) U.S. Cl. 514/255.06; 514/365; 514/409; 544/406; 548/147; 548/409

(57) ABSTRACT

This invention relates to the compounds of formula (I) shown below. Each variable in formula (I) is defined in the specification. These compounds can be used to treat hepatitis C virus infection.

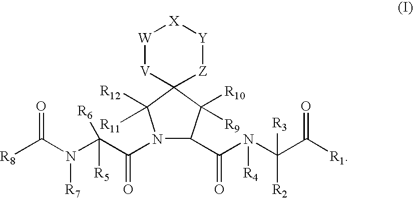

### CROSS-REFERENCE TO RELATED APPLICATIONS

This application claims priority from U.S. Provisional Patent Application No. 60/856,231, filed Nov. 2, 2006. The contents of the prior application are incorporated herein by reference in their entireties.

### BACKGROUND

Hepatitis C virus (HCV) is a (+)-sense single-stranded RNA virus that has been implicated as the major causative agent for most cases of non-A, non-B hepatitis. HCV has been implicated in liver cirrhosis and induction of hepatocellular carcinoma. Infection by HCV is a compelling human health problem. See, e.g., WO 05/007681; WO 89/04669; EP 381216; Alberti et al., J. Hepatology, 31 (Suppl. 1), 17-24 (1999); Alter, J. Hepatology, 31 (Suppl. 1), 88-91 (1999); and Lavanchy, J. Viral Hepatitis, 6, 35-47 (1999).

A HCV protease necessary for viral replication contains about 3000 amino acids. It includes a nucleocapsid protein (C), envelope proteins (E1 and E2), and several non-structural proteins (NS2, NS3, NS4a, NS5a, and NS5b).

NS3 protein possesses serine protease activity and is considered essential for viral replication and infectivity. The essentiality of the NS3 protease was inferred from the fact that mutations in the yellow fever virus NS3 protease decreased viral infectivity. See, e.g., Chamber et al., Proc. Natl. Acad. Sci. USA 87, 8898-8902 (1990). It was also demonstrated that mutations at the active site of the HCV NS3 protease completely inhibited the HCV infection in chimpanzee model. See, e.g., Rice et al., J. Virol. 74 (4) 2046-51 (2000). Further, the HCV NS3 serine protease was found to facilitate proteolysis at the NS3/NS4a, NS4a/NS4b, NS4b/NS5a, NS5a/NS5b junctions and was thus responsible for generating four viral proteins during viral replication. See, e.g., US 2003/0207861. Consequently, the HCV NS3 serine protease enzyme is an attractive target in treating HCV infection. Potential NS3 HCV protease inhibitors can be found in WO 02/18369, WO 00/09558, WO 00/09543, WO 99/64442, WO 99/07733, WO 99/07734, WO 99/50230, WO 98/46630, WO 98/17679, WO 97/43310, U.S. Pat. No. 5,990,276, Dunsdon et al., Biorg. Med. Chem. Lett. 10, 1571-1579 (2000); Llinas-Brunet et al., Biorg. Med. Chem. Lett. 10, 2267-2270 (2000); and S. LaPlante et al., Biorg. Med. Chem. Lett. 10, 2271-2274 (2000).

Due to lack of immunity or remission associated with HCV infection, hepatitis caused by HCV infection is more difficult to treat comparing to other forms of hepatitis. The only anti-HCV therapies currently available are interferon-α, interferon-α/ribavirin combination, and pegylated interferon-α. However, sustained response rates for interferon-α or interferon-α/ribavirin combination were found to be <50% and patients suffer greatly from side effects of these therapeutic agents. See, e.g., Walker, DDT, 4, 518-529 (1999); Weiland, FEMS Microbial. Rev., 14, 279-288 (1994); and WO 02/18369. Thus, there remains a need for developing more effective and better-tolerated therapeutic drugs.

### SUMMARY

This invention is based on the unexpected discovery that certain peptide-like compounds are effective in treating hepatitis C virus (HCV) infection by inhibiting hepatitis C viral proteases.

In one aspect, this invention features a compound of formula (I):

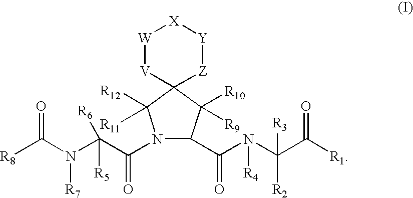

In formula (I), each of V, W, X, Y, and Z, independently, is O, S, S(O), S(O)₂, C(Rₐ₁Rₐ₂), C(O), N(Rₐ₁), or deleted; or V and W, W and X, X and Y, or Y and Z, together are aryl, C₃-C₂₀ cycloalkyl, or C₁-C₂₀ heterocycloalkyl; provided that at least one of V, W, X, Y, and Z is C(O), at most one of V, W, X, Y, and Z is deleted, and at most two of V, W, X, Y, and Z are O, S, S(O), S(O)₂, C(O), or N(Rₐ₁); R₁ is H, OR_{b1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C(O)-N(R_{b1}R_{b2}), N(R_{b1})-C(O)R_{b2}, N(R_{b1}R_{b2}), or N(R_{b1})-S(O)₂Rb₂; each of R2 and R3, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or, heteroaryl; or R₂ and R₃, together with the carbon atom to which they are attached, are C₃-C₂₀ cycloalkyl or C₁-C₂₀ heterocycloalkyl; each of R₅ and R₆, independently, is H, C₁-C₁₀ alkyl, C₂-C₁₀ alkenyl, C₂-C₁₀ alkynyl, C_{3 -C}₂₀ cycloalkyl, C₃-C₂₀ cycloalkenyl, C₁-C₂₀ heterocycloalkyl, C₁-C₂₀ heterocycloalkenyl, aryl, or, heteroaryl; R₈ is OR_{c1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, N(R_{c1}R_{c2}), or N(R_{c1})—C(O)—N(R_{c2}R_{c3}); and each of R₄, R₇, R₉, R₁₀, R₁₁, and R₁₂, independently, is H or C₁-C₁₀ alkyl; in which each of Rₐ₁, Rₐ₂, R_{b1}, R_{b2}, R_{b3}, R_{c1}, R_{c2}, and R_{c3} independently, is H, halo, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or heteroaryl; or Rₐ₁ and Rₐ₂, together with the atom to which they are attached, are C₃-C₂₀ cycloalkyl or C₁-C₂₀ heterocycloalkyl.

Referring to formula (I), a subset of the compounds described above are those in which at least one of V and W, W and X, X and Y, or Y and Z, taken together, is aryl, or C₁-C₂₀ heterocycloalkyl optionally substituted with OR or N(R)—C(O)R′; the remaining V, W, X, Y, and Z, independently, is O, S, C(Rₐ₁Rₐ₂), C(O), N(Rₐ₁), or deleted; in which each of R and R′, independently, is H or C₁-C₁₀ alkyl optionally substituted with aryl or C₂-C₁₀ alkenyl, and each of Rₐ₁ and Rₐ₂, independently, is H, C₂-C₁₀ alkenyl, C₂-C₁₀ alkynyl, or C₁-C₁₀ alkyl optionally substituted with aryl. In these compounds, R₁ can be OR_{b1}, C(O)—N(R_{b1}R_{b2}), or N(R_{b1})—S(O)₂R_{b2}, in which each of R_{b1} and R_{b2}, independently, is H, C₁-C₁₀ alkyl, C₃-C₂₀ cycloalkyl, C₁-C₂₀ heterocycloalkyl, aryl, or heteroaryl; each of R₂ and R₃, independently, can be H or C₁-C₁₀ alkyl; or R₂ and R₃, together with the carbon atom to which they are attached, can be C₃-C₂₀ cycloalkyl substituted with C₂-C₁₀ alkenyl; each of R₅ and R₆, independently, is H or isobutyl; and R₈ can be OR_{c1}, N(R_{c1}R_{c2}), N(R_{c1})—C(O)—N(R_{c2}R_{c3}), or C₁-C₁₀ alkyl substituted with C₃-C₂₀ cycloalkyl, or N(R_{c1})—C(O)R_{c2} in which each of R_{c1}, R_{c2}, and R_{c3}, independently, is H, C₁-C₁₀ alkyl, C₃-C₂₀ cycloalkyl, C₁-C₂₀ heterocycloalkyl, aryl, or heteroaryl. For example, R₈ can be

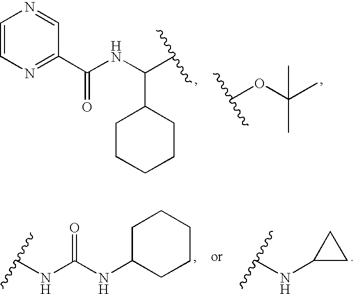

Referring to formula (I), another subset of the compounds described above are those in which each of V, W, X, Y, and Z, independently, is O, S, C(Rₐ₁Rₐ₂), C(O), N(Rₐ₁), or deleted; in which each of Rₐ₁ and Rₐ₂, independently, is H or C₁-C₁₀ alkyl. In these compounds, R₁ can be C(O)—N(R_{b1}R_{b2}), in which each of R_{b1} and R_{b2}, independently, is H, C₁-C₁₀ alkyl, C₃-C₂₀ cycloalkyl, C₁-C₂₀ heterocycloalkyl, aryl, or heteroaryl; each of R₂ and R₃, independently, can be H or C₁-C₁₀ alkyl optionally substituted with C₃-C₂₀ cycloalkyl; each of R₅ and R₆, independently, can be H or isobutyl; and R₈ can be OR_{c1}, or C₁-C₁₀ alkyl substituted with C₃-C₂₀ cycloalkyl, N(R_{c1})—C(O)R_{c2},or N(R_{c1})—C(O)—N(R_{c2}R_{c3}); in which each of R_{c1}, R_{c2}, and R_{c3}, independently, is H, C₁-C₁₀ alkyl, C₃-C₂₀ cycloalkyl, C₁-C₂₀ heterocycloalkyl, aryl, or heteroaryl. For

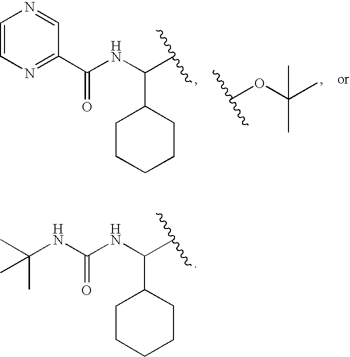

example, R₈ can be

Referring to formula (I), still another subset of the compounds described above

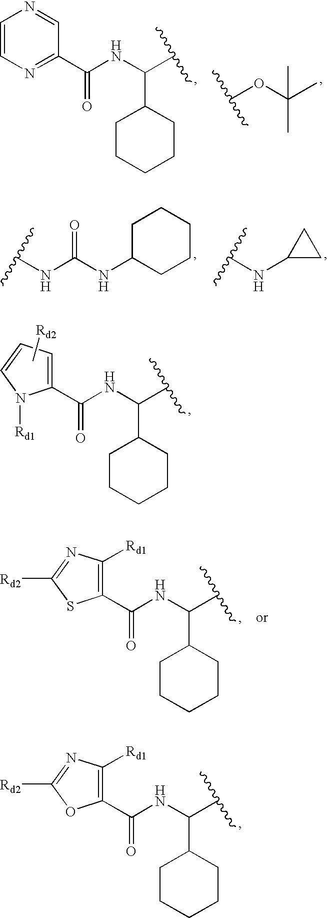

are those in which R₈ is
in which each of R_{d1} and R_{d2}, independently, is H or C₁-C₁₀ alkyl; R₁ can be OR_{b1}, C(O)—N(R_{b1}R_{b2}), or N(R_{b1})—S(O)₂Rb₂, in which each of R_{b1} and R_{b2}, independently, is H, C₁-C₁₀ alkyl, C₃-C₂₀ cycloalkyl, C₁-C₂₀ heterocycloalkyl, aryl, or heteroaryl; each of R₂ and R₃, independently, can be H or C₁-C₁₀ alkyl; or R₂ and R₃, together with the carbon atom to which they are attached, are C₃-C₂₀ cycloalkyl substituted with C₂-C₁₀ alkenyl; and each of R₅ and R₆, independently, can be H or isobutyl.

The term “alkyl” refers to a saturated, linear or branched hydrocarbon moiety, such as —CH₃ or —CH(CH₃)₂. The term “alkenyl” refers to a linear or branched hydrocarbon moiety that contains at least one double bond, such as —CH═CH—CH₃. The term “alkynyl” refers to a linear or branched hydrocarbon moiety that contains at least one triple bond, such as —C≡C—CH₃. The term “cycloalkyl” refers to a saturated, cyclic hydrocarbon moiety, such as cyclohexyl. The term “cycloalkenyl” refers to a non-aromatic, cyclic hydrocarbon moiety that contains at least one double bond, such as cyclohexenyl. The term “heterocycloalkyl” refers to a saturated, cyclic moiety having at least one ring heteroatom (e.g., N, O, or S), such as 4-tetrahydropyranyl. The term “heterocycloalkenyl” refers to a non-aromatic, cyclic moiety having at least one ring heteroatom (e.g., N, O, or S) and at least one ring double bond, such as pyranyl. The term “aryl” refers to a hydrocarbon moiety having one or more aromatic rings. Examples of aryl moieties include phenyl (Ph), phenylene, naphthyl, naphthylene, pyrenyl, anthryl, and phenanthryl. The term “heteroaryl” refers to a moiety having one or more aromatic rings that contain at least one heteroatom (e.g., N, O, or S). Examples of heteroaryl moieties include furyl, furylene, fluorenyl, pyrrolyl, thienyl, oxazolyl, imidazolyl, thiazolyl, pyridyl, pyrimidinyl, quinazolinyl, quinolyl, isoquinolyl and indolyl.

Alkyl, alkenyl, alkynyl, cycloalkyl, cycloalkenyl, heterocycloalkyl, heterocycloalkenyl, aryl, and heteroaryl mentioned herein include both substituted and unsubstituted moieties, unless specified otherwise. Possible substituents on cycloalkyl, cycloalkenyl, heterocycloalkyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, nitroso, azido, acyl, thioacyl, acyloxy, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

In another aspect, this invention features a method for treating HCV infection. The method includes administering to a subject in need thereof an effective amount of one or more compounds of formula (I) shown above. The term “treating” or “treatment” refers to administering one or more compounds of formula (I) to a subject, who has a HCV infection, a symptom of it, or a predisposition toward it, with the purpose to confer a therapeutic effect, e.g., to cure, relieve, alter, affect, ameliorate, or prevent the HCV infection, the symptom of it, or the predisposition toward it.

In addition, this invention encompasses a pharmaceutical composition that contains an effective amount of at least one of the compounds of formula (I) and a pharmaceutically acceptable carrier. The composition can further include a second antiviral agent, such as ribavirin or interferon. Examples of interferon include α-interferon or pegylated interferon. The term “pegylated interferon” mentioned herein refers to an interferon that contains a polyethylene glycol moiety.

The compounds of formula (I) described above include the compounds themselves, as well as their salts, prodrugs, and solvates, if applicable. A salt, for example, can be formed between an anion and a positively charged group (e.g., amino) on a compound of formula (I). Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a compound of formula (I). Suitable cations include sodium ion, potassium ion, magnesium ion, calcium ion, and an ammonium cation such as tetramethylammonium ion. The compounds of formula (I) also include those salts containing quaternary nitrogen atoms. Examples of prodrugs include esters and other pharmaceutically acceptable derivatives, which, upon administration to a subject, are capable of providing active compounds of formula (I). A solvate refers to a complex formed between an active compound of formula (I) and a pharmaceutically acceptable solvent. Examples of pharmaceutically acceptable solvents include water, ethanol, isopropanol, ethyl acetate, acetic acid, and ethanolamine.

Also within the scope of this invention is a composition containing one or more of the compounds of formula (I) described above for use in treating a HCV infection, and the use of such a composition for the manufacture of a medicament for the just-mentioned treatment.

The details of one or more embodiments of the invention are set forth in the description below. Other features, objects, and advantages of the invention will be apparent from the description and from the claims.

### DETAILED DESCRIPTION

Shown below are 48 exemplary compounds of this invention.

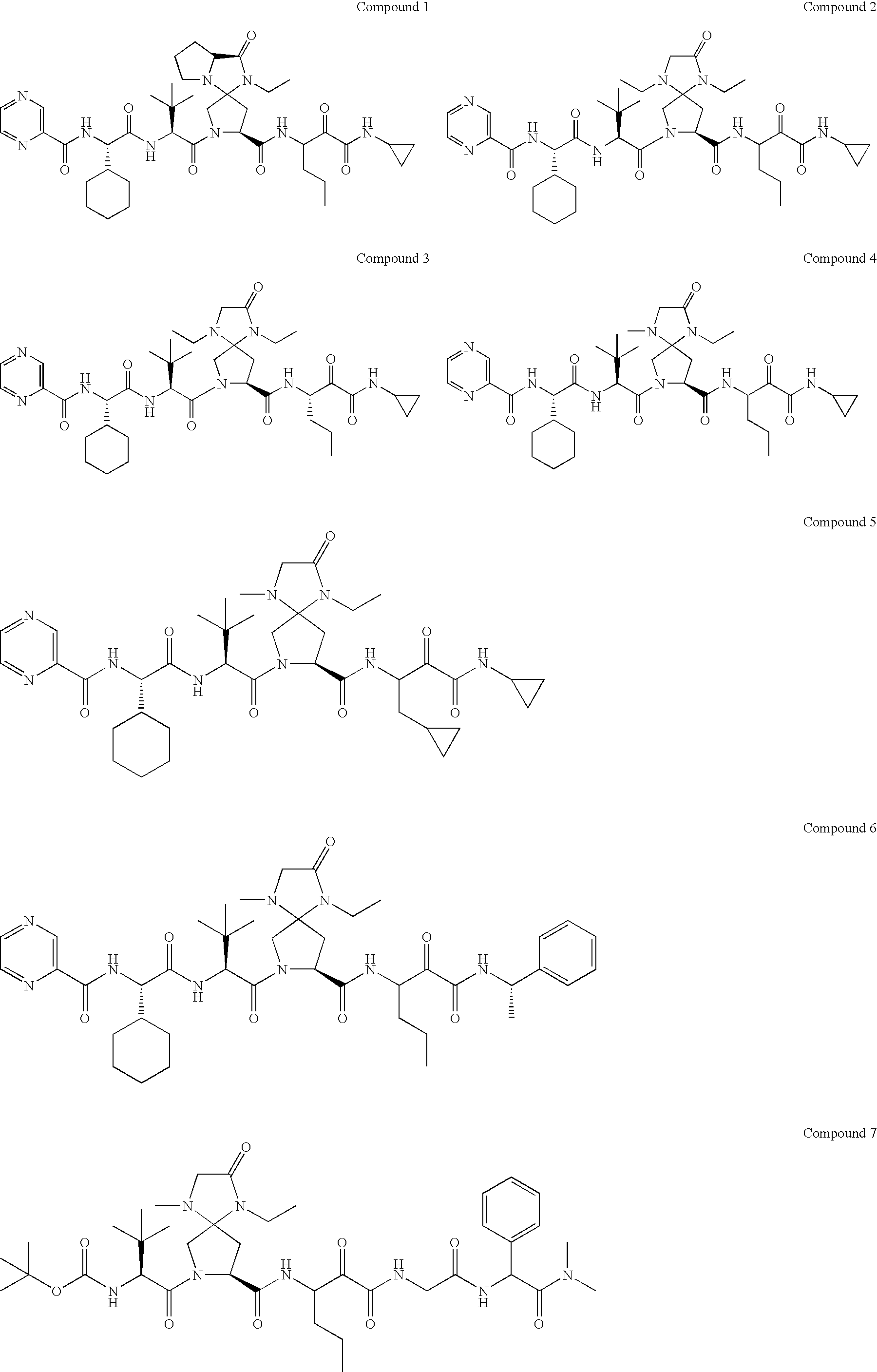

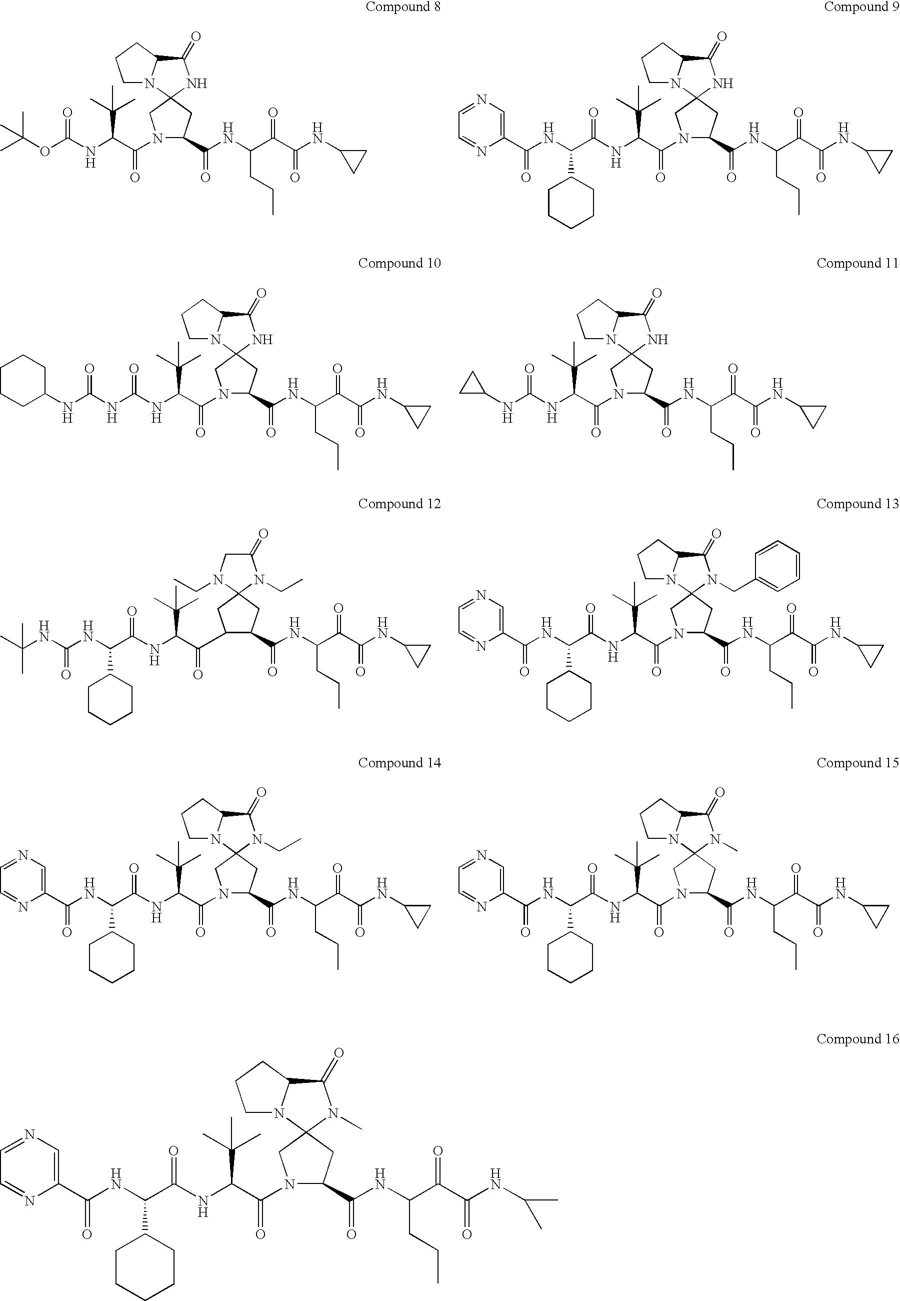

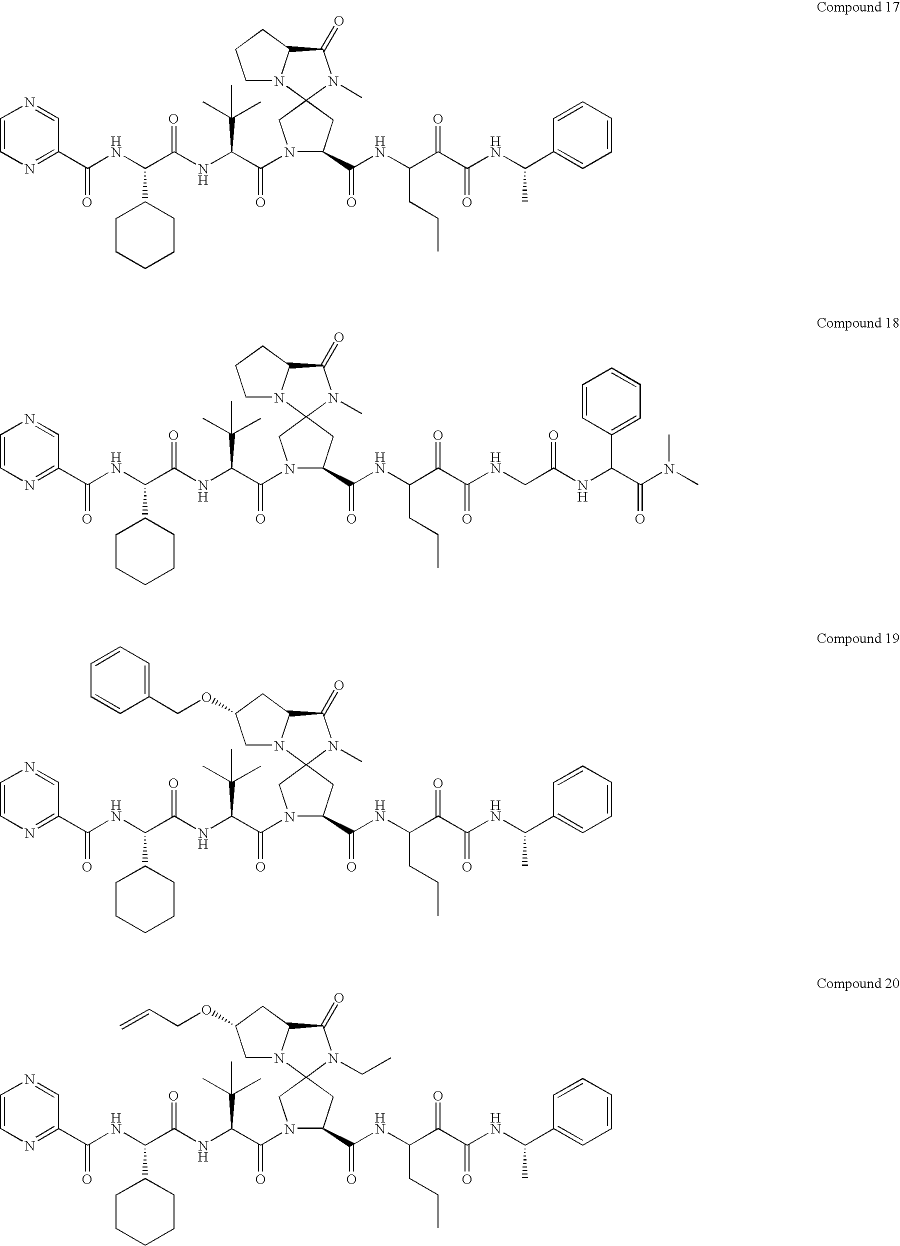

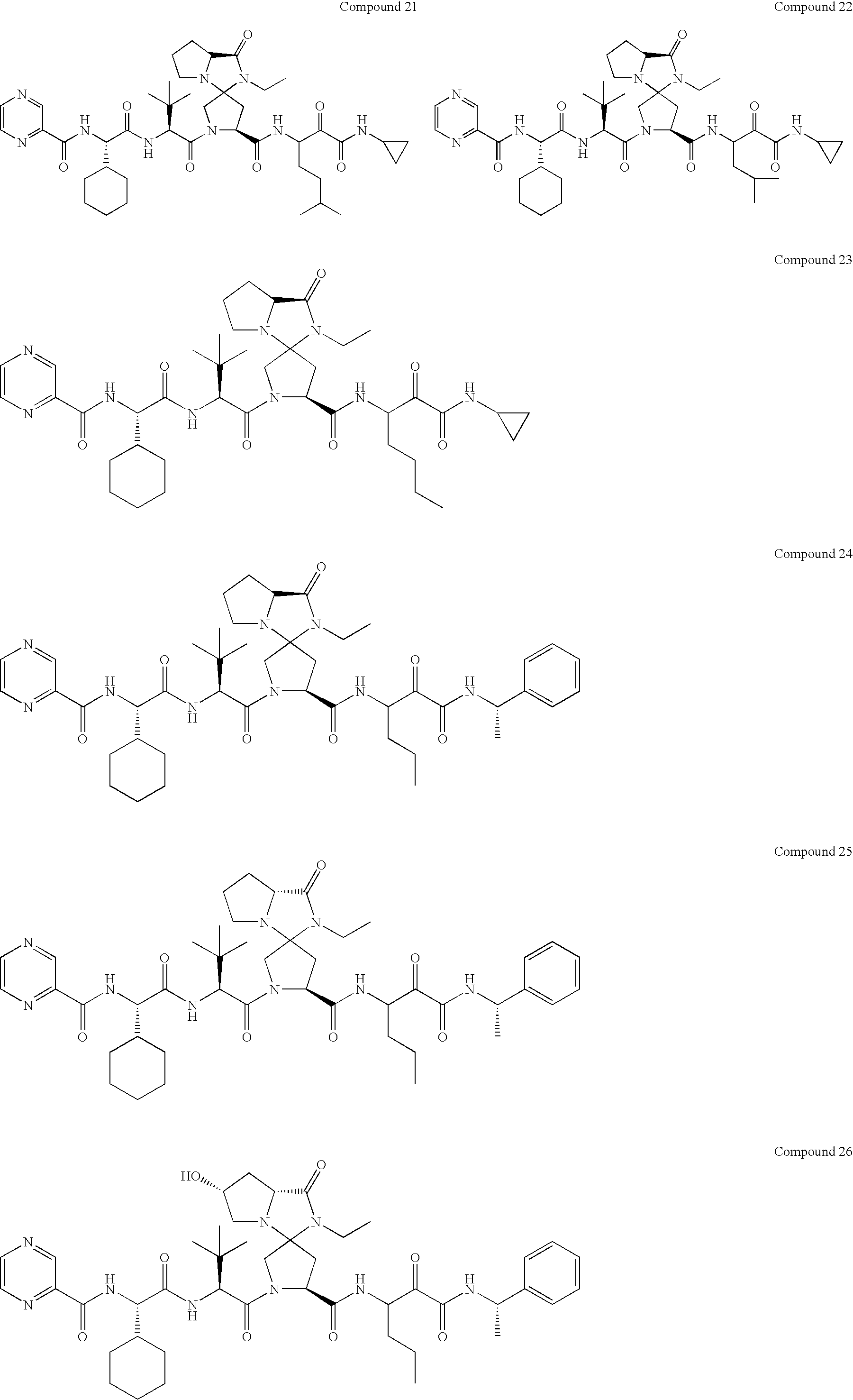

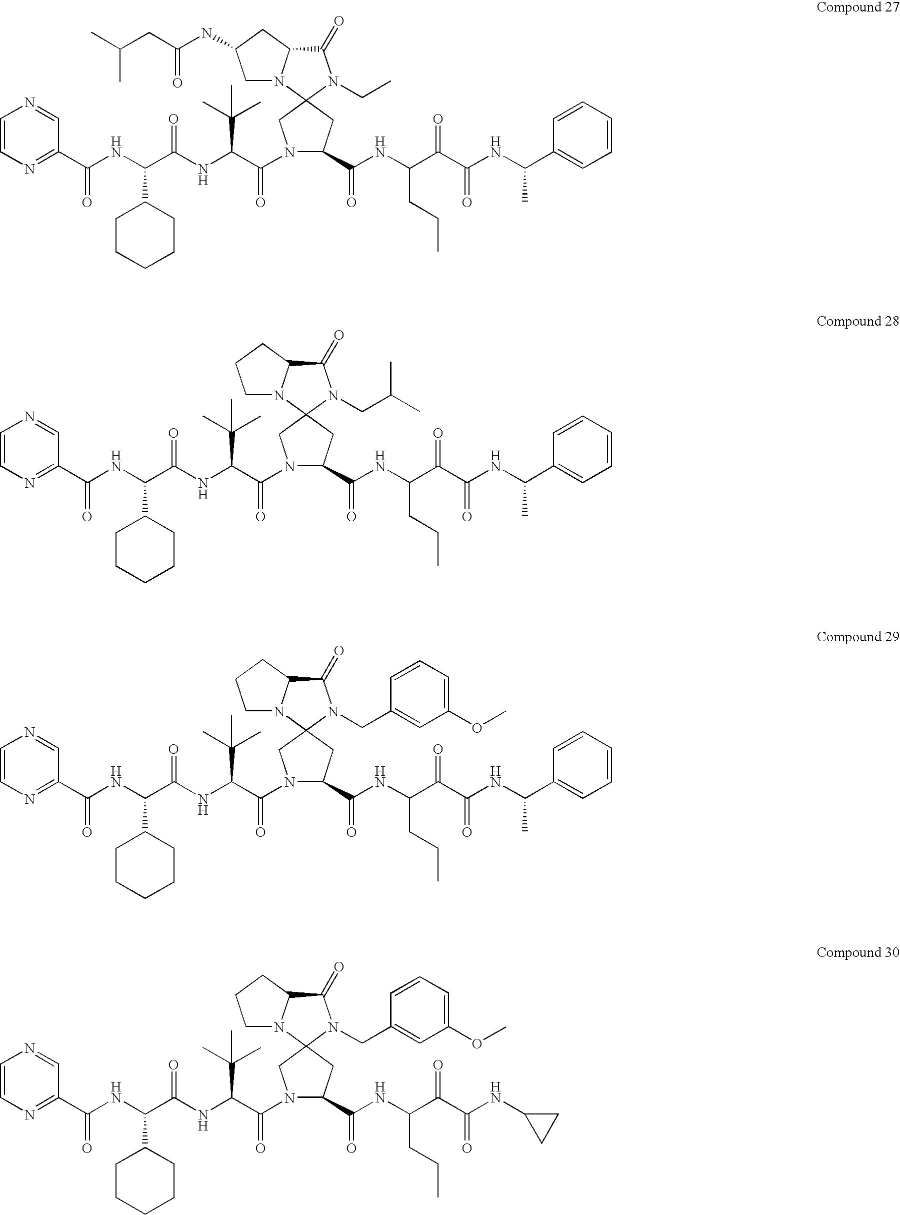

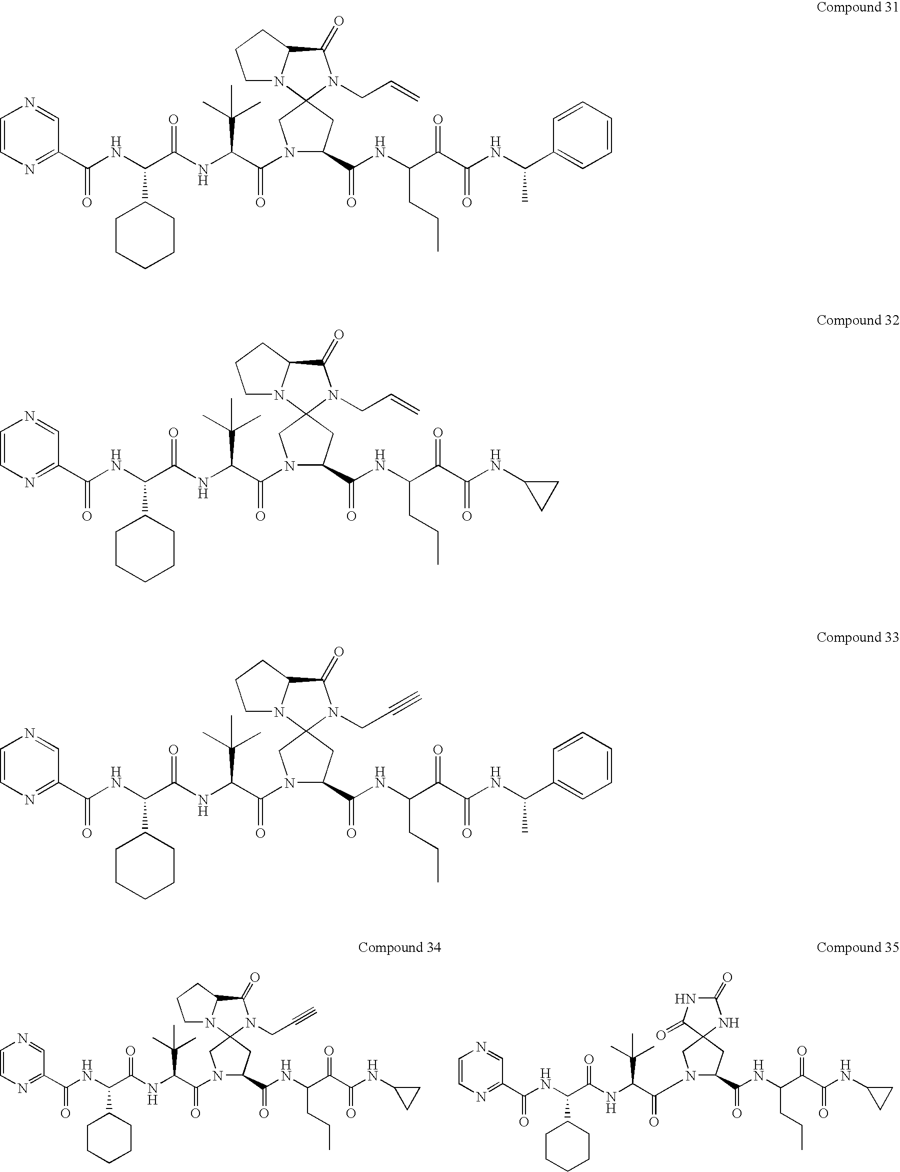

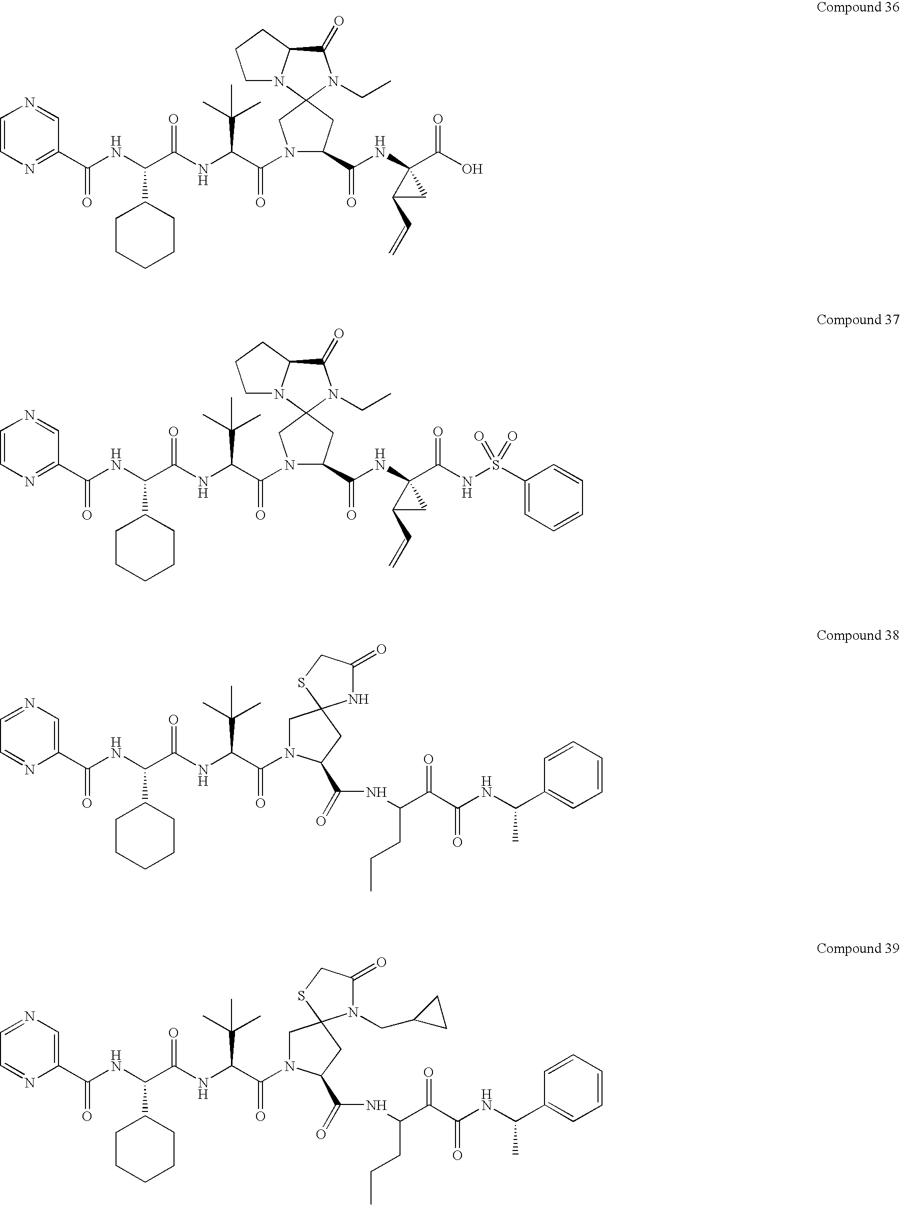

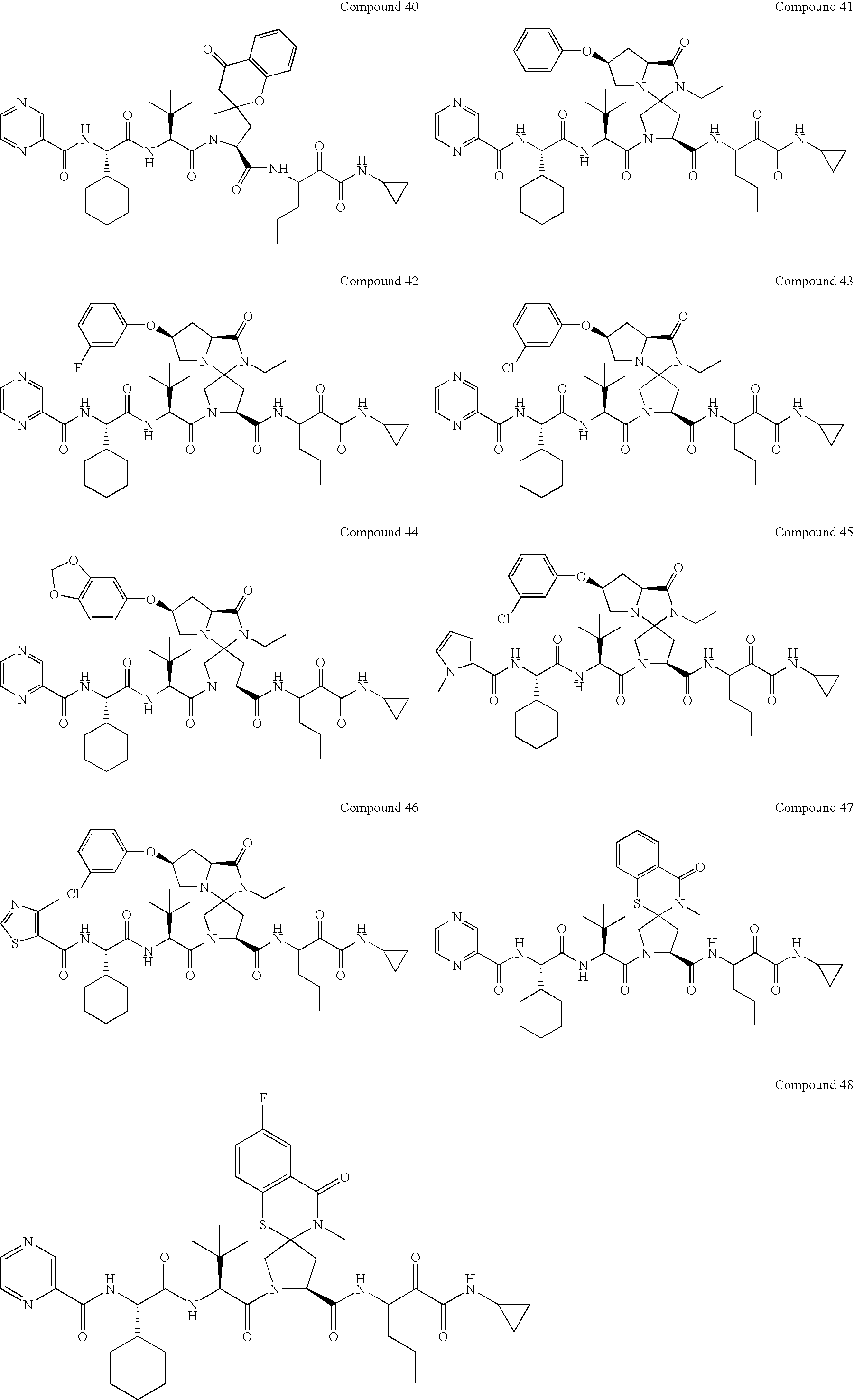

The compounds of formula (I) described above can be prepared by methods well known in the art. Examples 1-48 below provide detailed descriptions of how compounds 1-48 were actually prepared.

Scheme 1 shown below illustrates a typical route for synthesizing certain exemplary compounds. Specifically, commercially available (S)-1-t-butyl 2-methyl 4-oxopyrrolidine-1,2-dicarboxylate (i.e., compound A) can first react with a diamine compound (e.g., compound B) to form a triazaspirononyl-containing compound (e.g., compound C). The t-Boc group can be removed to form a deprotected compound (e.g., compound D), which can then react with 2-tert-butoxycarbonylamino-3,3-dimethyl-butyric acid to form a di-amide (e.g., compound E). The t-Boc group in the di-amide can again be removed to form a deprotected compound (e.g., compound F), which can then react with an acid to form another di-amide (e.g., compound G). The methyl carboxylate group on the pyrrolidine ring in the di-amide thus obtained can be hydrolyzed to form an acid (e.g., compound H). The acid can subsequently react with a hydroxyl-containing amine (e.g., compound I) to form a tri-amide (e.g., compound J), which can be reduced to form certain compounds of this invention (e.g., compounds 1-34 and 41-46).

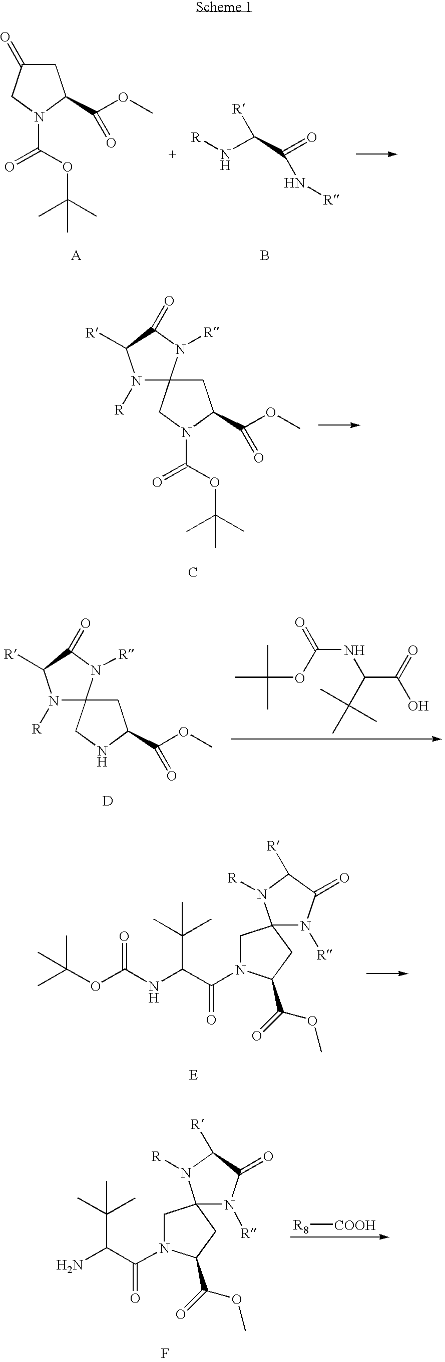

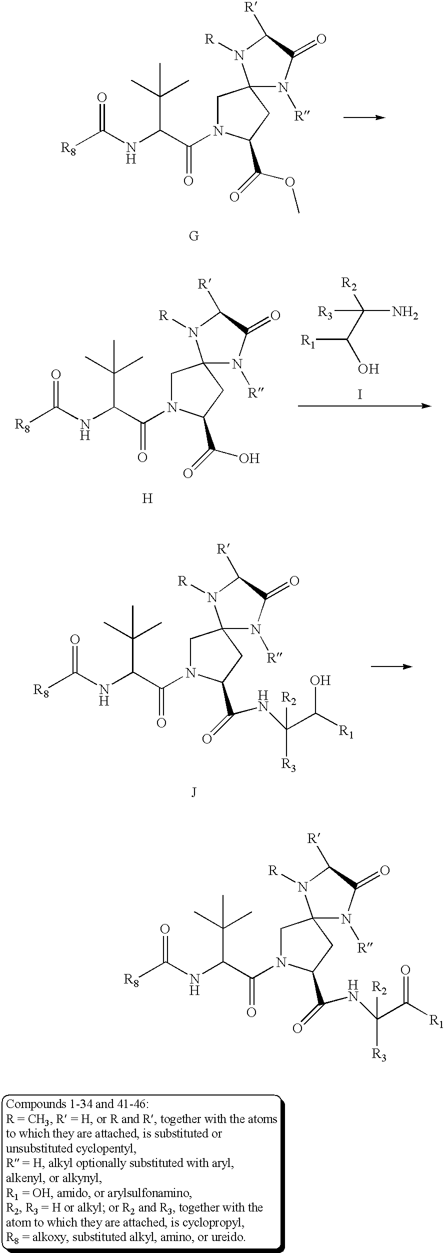

The intermediates mentioned in Scheme 1 above can be obtained through other synthetic routes. For example, compound G can be prepared by the method illustrated in Scheme 2 below. Specifically, commercially available (2S,4R)-1-tert-butyl 2-methyl 4-hydroxypyrrolidine-1,2-dicarboxylate can first be deprotected to remove the t-Boc group. The compound thus obtained (e.g., compound K) can react with 2-tert-butoxycarbonylamino-3,3-dimethyl-butyric acid to form a di-amide (e.g., compound L). The t-Boc group in the di-amide thus obtained can again be removed to form a deprotected compound (e.g., compound M), which can then react with an acid to form another di-amide (e.g., compound N). The hydroxyl group in the di-amide thus obtained can be oxidized to form a pyrrolidinone compound (e.g., compound O), which can then react with a diamine compound to form compound G.

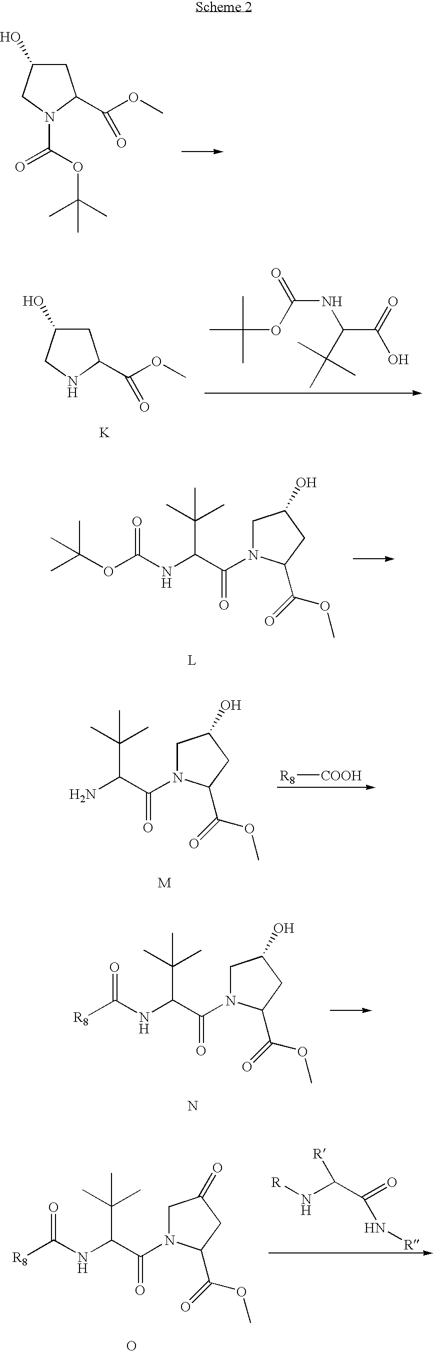

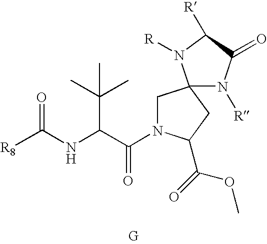

The intermediates mentioned in Schemes 1 and 2 above can be modified to prepare other compounds of this invention. For example, compound A can react with ammonium carbonate to form a triazaspirononyl-containing compound in which the triazaspirononyl ring has two carbonyl group. See Scheme 7 and Example 35. The compound thus obtained can then be used to prepare compound 35 in a manner similar to that illustrated in Scheme 1. As another example, compound H can react with 1-amino-2-vinyl-cyclopropanecarboxylic acid methyl ester to form an intermediate, which can be converted to compound 36 and 37. See Scheme 8 and Examples 36 and 37. As another example, compound O can react with an amine compound having a thiol group to form a compound containing thiadiazaspirononyl ring, which can then be used to prepare compounds 38 and 39 in a manner similar to that illustrated in Scheme 1. See Schemes 9 and 10, and Examples 38, 39, 47, and 48. As a further example, compound O can react with 2-hydroxyacetophenone to form a compound containing a spiro(chroman- 2,3′-pyrrolidin)-4-one ring, which can then be used to prepare compound 40 in a manner similar to that illustrated in Scheme 1. See Scheme 11 and Example 40.

A compound synthesized above can be purified by a suitable method such as column chromatography, high-pressure liquid chromatography, or recrystallization.

Other compounds of formula (I) can be prepared using other suitable starting materials through the above synthetic routes and others known in the art. The methods described above may also additionally include steps, either before or after the steps described specifically herein, to add or remove suitable protecting groups in order to ultimately allow synthesis of the compounds of formula (I). In addition, various synthetic steps may be performed in an alternate sequence or order to give the desired compounds. Synthetic chemistry transformations and protecting group methodologies (protection and deprotection) useful in synthesizing applicable compounds of formula (I) are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The compounds mentioned herein may contain a non-aromatic double bond and one or more asymmetric centers. Thus, they can occur as racemates and racemic mixtures, single enantiomers, individual diastereomers, diastereomeric mixtures, tautomers, and cis- or trans- isomeric forms. All such isomeric forms are contemplated.

Also within the scope of this invention is a pharmaceutical composition containing an effective amount of at least one compound of formula (I) described above and a pharmaceutical acceptable carrier. Further, this invention covers a method of administering an effective amount of one or more of the compounds of formula (I) to a patient having a HCV infection. “An effective amount” refers to the amount of an active compound of formula (I) that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more compounds of formula (I) can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more active compounds of formula (I) can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound of formula (I). Examples of other carriers include colloidal silicon oxide, magnesium stearate, cellulose, sodium lauryl sulfate, and D&C Yellow # 10.

The compounds of formula (I) described above can be preliminarily screened for their efficacy in treating HCV infection by an in vitro assay (Example 41 below) and then confirmed by animal experiments and clinic trials. Other methods will also be apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-1′-oxohexahydrospiro[pyrrolidine-3,3′-pyrrolo[1,2-c]imidazole]-5-carboxamide

Compound 1 was prepared by two methods, i.e., methods A and B. Method A is illustrated in Scheme 3 below.

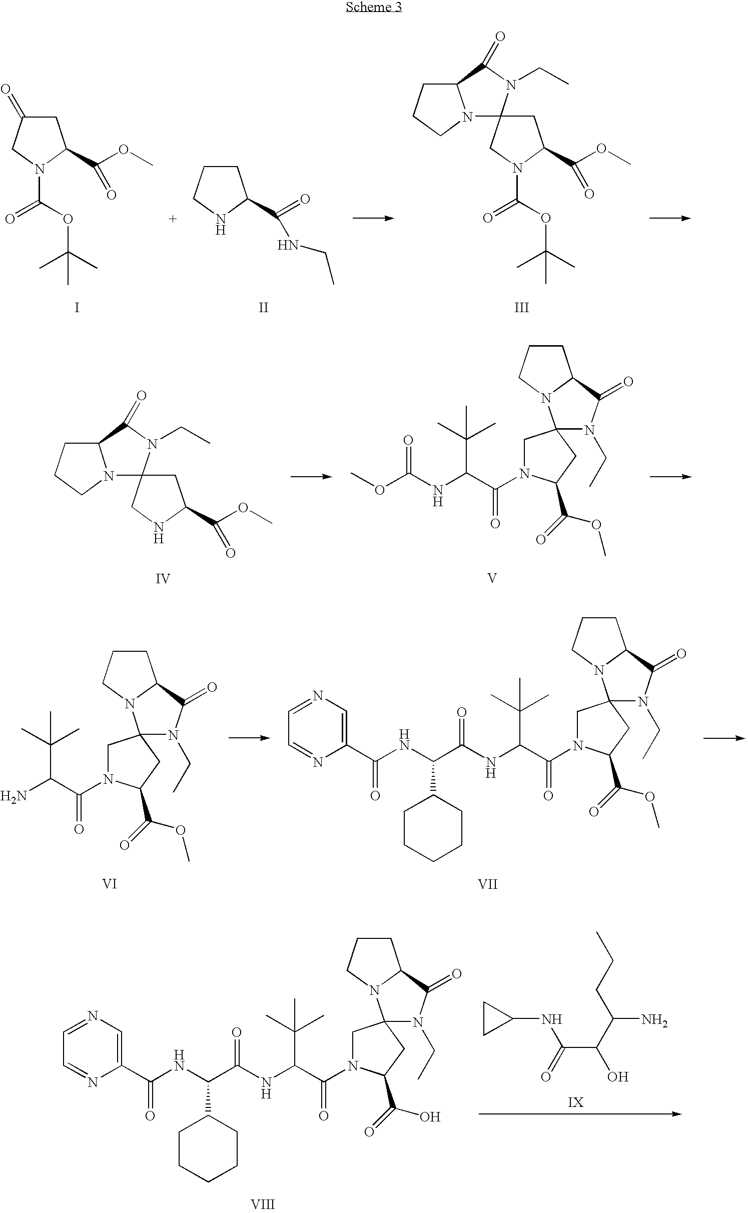

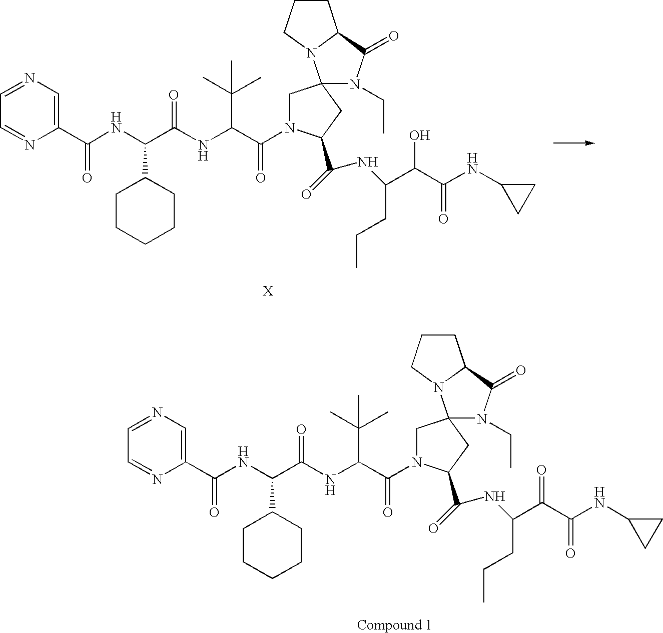

A solution of compound I (400 mg, 1.64 mmol), compound II (462 mg, 2.08 mmol), and MgSO₄ (2 g) in methanol (60 mL) was refluxed overnight. Compounds I and II are commercially available. The reaction mixture was then filtered, concentrated, and purified using silica gel chromatography to give 0.24 g (40% yield) of intermediate III (see also J. Comb. Chem. 2003, 5, 356). LC/MS: 367.8 (M+H)⁺.

To a solution of intermediate III (200 mg, 0.54 mmol) in CH₂Cl₂ (30 mL) was added a solution of 4 N HCl in dioxane (10 mL). The reaction mixture was stirred at room temperature for 3 hours. The mixture was then concentrated to afford a quantitative yield of crude intermediate IV, which was used in the next step without further purification.

N-methyl morphorline (1 mL) was added to a solution of 2-tert-butoxycarbonylamino-3,3-dimethyl-butyric acid (231.3 mg, 1.0 mmol), 1-ethyl-3-(3-dimethyllaminopropyl)carbodiimide hydrochloride (EDC, 395.4 mg, 1.5 mmol), Bu^{t}OH (135.1 mg, 1.0 mmol) and intermediate IV (267 mg, 1.0 mmol) in CH₂Cl₂ (30 mL) at room temperature. After the reaction mixture was stirred at that same temperature overnight, it was quenched with water. The mixture was then extracted with CH₂Cl₂ (60 mL). The organic layer was collected, dried, concentrated, and purified using silica gel chromatography to afford 288 mg (yield: 60%) of intermediate V. LC/MS: 481.3 (M+H)⁺.

To a solution of intermediate V (240 mg, 0.5 mmol) in CH₂Cl₂ (30 mL) was added a solution of 4 N HCl in dioxane (10 mL). The reaction mixture was stirred at room temperature for 3 hours and then concentrated by vacuum to afford a quantitative yield of crude intermediate VI, which was used in the next step without further purification. LC/MS: 381.2 (M+H)⁺.

N-methyl morphorline (1 mL) was added to a solution of 2-tert-butoxycarbonylamino-3,3-dimethyl-butyric acid (263 mg, 1.0 mmol), EDC (296.6 mg, 1.5 mmol), Bu^{t}OH (135.1 mg, 1.0 mmol) and intermediate VI (380.5 mg, 1.0 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified using silica gel chromatography to afford 407 mg (yield: 65%) of intermediate VII. LC/MS: 626.1 (M+H)⁺.

To a solution of intermediate VII (400 mg, 0.64 mmol) in THF (30 mL) was added a solution of 0.5 M LiOH (10 mL). After the resulting mixture was stirred at room temperature for 3 hours, it was neutralized to pH of 5˜6 with 1N HCl. The reaction solution was then extracted with CH₂Cl₂ (100 mL). The organic layer was collected and concentrated to afford a crude intermediate VIII, which was used in next step without further purification. LC/MS: 612.1 (M+H)⁺.

N-methyl morphorline (1 mL) was added to a solution of intermediate VIII (183.5 mg, 0.3 mmol), EDC (89.0 mg, 0.45 mmol), Bu^{t}OH (40.5 mg, 0.3 mmol), and 3-Amino-2-hydroxy-hexanoic acid cyclopropylamide (compound IX, 55.8 mg, 0.3 mmol) in CH₂Cl₂ (30 mL) at room temperature. After the reaction mixture was stirred at that temperature overnight, it was quenched with water. The mixture was then extracted with CH₂Cl₂ (60 mL). The organic layer was collected, dried, concentrated, and purified using silica gel chromatography to afford 164 mg (yield: 70%) of compound X. LC/MS: 780.4 (M+H)⁺.

To a solution of intermediate X (156 mg, 0.2 mmol) in CH₂Cl₂ (30 mL) was added Dess-Martin reagent (156 mg, 0.36 mmol) at room temperature. After the reaction mixture was stirred at the same temperature for about 3 hours, it was quenched with a 1N NaOH aqueous solution (5 mL). The mixture was then extracted with CH₂Cl₂ (50 mL). The organic layer was collected, concentrated, and purified using silica gel chromatography to afford 140 mg (yield: 90%) of compound 1 (see J. Org. Chem. 1983, 48, 4155). LC/MS: 778.2 (M+H)⁺; 810.2 (M+H+MeOH)⁺; 832.2 (M+Na+MeOH)⁺. ¹H NMR (CDCl₃): δ 9.38 (s, 1H), 8.76 (s, 1H), 8.56 (s, 1H), 8.23 (d, J=8.7 Hz, 1H), 7.35-6.67 (m, 3H), 5.40-5.23 (m, 1H), 4.82-4.41 (m, 3H), 4.01-2.72 (m, 8H), 2.39-0.72 (m, 40H)

Compound II mentioned above was prepared by the method illustrated in Scheme 4 below:

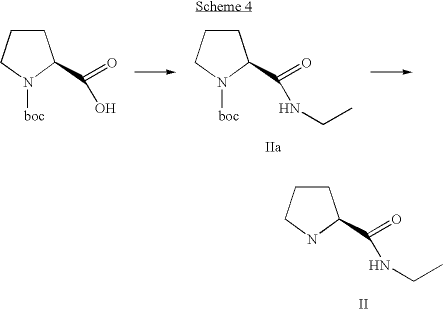

N-methyl morphorline (2.0 mL) and DMF (5 mL) were added to a solution of commercially available pyrrolidine-1,2-dicarboxylic acid 1-tert-butyl ester (2.15 g, 10.0 mmol), EDC (2.96 g, 15.0 mmol), Bu^{t}OH (1.35 g, 10.0 mmol), and ethylamine hydrochloride (1.22 g, 15.0 mmol) in CH₂Cl₂ (150 mL) at room temperature. After the reaction mixture was stirred at the same temperature overnight, it was quenched with water. The mixture was then extracted with CH₂Cl₂. The organic layer was collected, dried over anhydrous MgSO₄, concentrated, and purified by silica gel chromatography to afford 1.94 g (yield 80%) of intermediate IIa.

To a solution of intermediate IIa (1.2 g, 5.0 mmol) in CH₂Cl₂ (50 mL) was added a solution of 4.0 N HCl in dioxane (10 mL). The reaction mixture was stirred at room temperature for 3 hours. The mixture was then concentrated to give a quantitative yield of the crude intermediate II, which was used to prepare intermediate III without further purification.

Intermediate IX mentioned above was prepared by the method illustrated in Scheme 5 below:

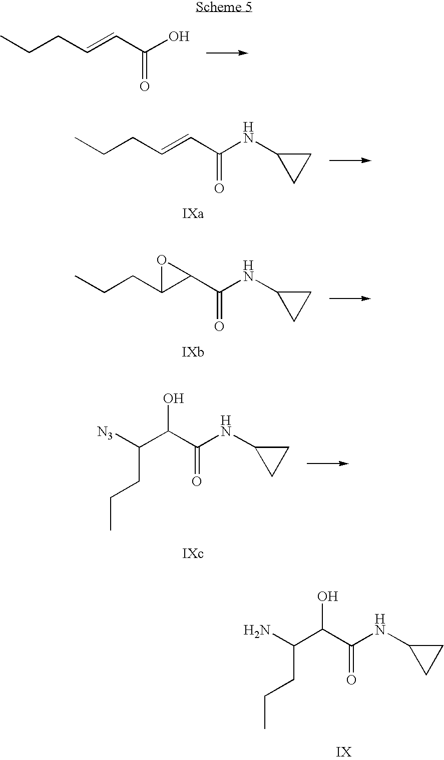

N-methyl morphorline (6.0 mL) was added to a solution of hex-2-enoic acid (11.4 g, 0.1 mol), EDC (29.6 g, 0.15 mol), Bu^{t}OH (13.5 g, 0.1 mol) and cyclopropylamine (5.7 g, 0.1 mol) in CH₂Cl₂ (300 mL) at room temperature. After the reaction mixture was stirred at the same temperature overnight, it was quenched with water. The mixture was then extracted with CH₂Cl₂. The organic layer was collected, dried over anhydrous MgSO₄, concentrated, and purified by silica gel chromatography to afford 12.2 g (yield: 80%) of intermediate IXa.

To a solution of tert-butyl hydroperoxide (1.42 g, 12.0 mmol) in dry THF (100 mL) was dropped 2.5 M BuLi (4.4 mL, 11.0 mmol) slowly at −78° C. for 1 hour. After the reaction mixture was stirred at that temperature for another 30 minutes, intermediate IXa (1.5 g, 10.0 mmol) in THF (30 mL) was added at the same temperature. The reaction mixture was warmed to room temperature and continually stirred overnight. It was then quenched with water and extracted with CH₂Cl₂. The organic layer was collected, dried over anhydrous MgSO₄, concentrated, and purified by silica gel chromatography to afford 12.2 g (yield: 80%) of intermediate IXb.

To a suspension of NaN₃ (0.7 g, 11.0 mmol) and MgSO₄ (1.4 g, 12 mmol) in methanol was added intermediate IXb (1.7 g, 10.0 mmol) at room temperature. The reaction mixture was refluxed overnight and then filtered. The filtrate was concentrated and purified by silica gel chromatography to afford 1.8 g (yield: 85%) of compound IXc.

To a solution of intermediate IXc (2.1 g, 10 mol) in methanol (100 mL) was added 5% Pd/C (50 mg) at room temperature. The reaction mixture was sequentially purged with N₂ and H₂. The reaction flask was kept under 30 psi of hydrogen gas at room temperature for 13 hours. The organic layer was then filtered and the filtrate was concentrated to afford 1.8 g (yield: 95%) of intermediate IX.

Method B is illustrated in Scheme 6 below:

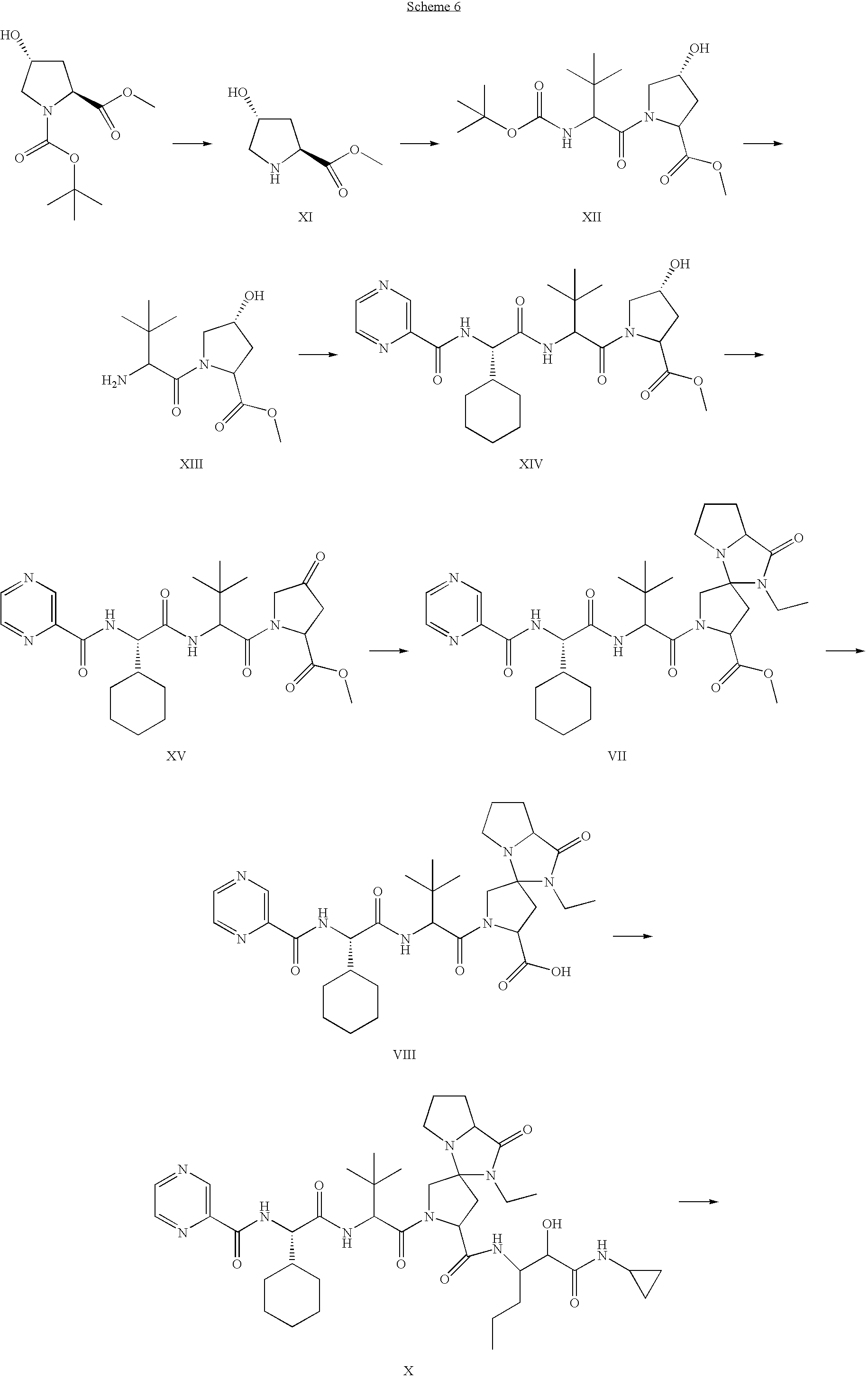

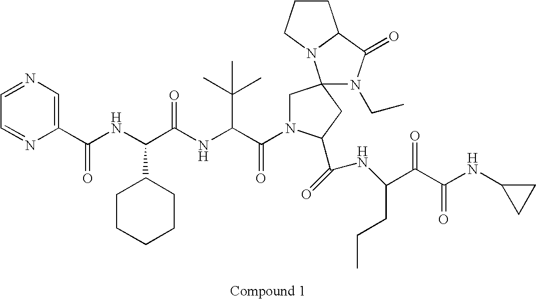

A solution of 4.0 N HCl in dioxane (50 mL) was added to a solution of commercially available 4-hydroxy-pyrrolidine-1,2-dicarboxylic acid 1-tert-butyl ester 2-methyl ester (30 g, 206 mmol) in dichloromethane (200 mL). After the reaction mixture was stirred at room temperature for 4 hours, it was concentrated to get a quantitative yield of crude intermediate XI, which was used in the next step without further purification.

N-methyl morphorline (5 mL) was added to a solution of intermediate XI (5.0 g, 34.4 mmol), EDC (10.2 g, 51.6 mmol), Bu^{t}OH (4.7 g, 34.4 mmol), and 2-tert-butoxycarbonylamino-3,3-dimethyl-butyric acid (8.0 g, 34.4 mmol) in CH₂Cl₂ (150 mL) at room temperature. After the reaction mixture was stirred at the same temperature overnight, it was quenched with water and extracted with CH₂Cl₂. The organic layer was then collected, dried with anhydrous MgSO₄, concentrated, and purified by silica gel chromatography to afford 7.4 g (yield: 60%) of intermediate XII. ¹H-NMR (CDCl₃): δ 5.30 (d, J=9.3 Hz, 1H), 4.46 (dd, J=8.4 Hz, J=Hz, 1H), 4.34 (s, 1H), 4.09-4.05 (m, 2H), 3.73-3.70 (m, 1H), 3.62-3.58 (m, 1H), 3.54 (s, 3H), 2.20-2.14 (m, 1H), 1.88-1.78 (m, 1H), 1.25 (s, 9H), 0.85 (s, 9H).

To a solution of intermediate XII (10 g, mmol) in CH₂Cl₂ (150 mL) was added a solution of 4.0 N HCl in dioxane (10 mL). After the reaction mixture was stirred at room temperature for 3 hours, it was concentrated to get a quantitative yield of crude intermediate XIII, which was used in the next step without further purification.

N-methyl morphorline (2.0 mL) and DMF (5 mL) were added to a solution of intermediate XIII (5.0 g, 19.3 mmol), EDC (7.0 g, 35.4 mmol), Bu^{t}OH (2.6 g, 19.3 mmol) and cyclohexyl-[(pyrazine-2-carbonyl)-amino]-acetic acid (5.1 g, 19.3 mmol) in CH₂Cl₂ (150 mL) at room temperature. After the reaction mixture was stirred at that temperature overnight, it was quenched with water. The mixture was the extracted with CH₂Cl₂. The organic layer was collected, dried with anhydrous MgSO₄, concentrated, and purified by silica gel by column chromatography to afford 4.9 g (yield: 50%) of intermediate XIV. ¹H-NMR (CDCl₃): δ 9.35 (d, J=1.2 Hz, 1H), 8.74 (d, J=2.15, 1H), 8.53 (m, 1H), 8.26 (d, 8.7 Hz, 1H), 6.60-6.67 (m, 1H), 4.62 (dd, J=8.7 Hz, J=8.7 Hz, 1H), 4.53-4.49 (m, 2H), 4.41 (dd, J=8.7 Hz, J=6.9 Hz, 1H), 4.04 (d, J=10.8 Hz, 1H), 3.70 (s, 3H), 2.39-2.31 (m, 1H), 2.01-1.10 (m, 15H), 1.01 (s, 9H).

To a solution of intermediate XIV (2.0 g, 3.97 mmol) and celite (2 g) in CH₂Cl₂ (100 mL) was added PCC (2.0 mg, 9.27 mmol) at room temperature. The reaction mixture was filtered after it was stirred at the same temperature for about 5 hours. The filtrate was concentrated and purified by silica gel chromatography to afford 1.1 g (yield: 55%) of intermediate XV. ¹H-NMR (CDCl3) δ 9.37 (s, 1H), 8.74 (d, J=2.1 Hz, 1H), 8.52 (s, 1H), 8.23 (d, J=8.7 Hz, 1H), 6.56 (d, J=8.4 Hz, 1H), 5.06 (dd, J=3.6 Hz, J=10.5 Hz, 1H), 4.43 (dd, J=8.7 Hz, J=17.4 Hz, 2H), 4.07 (d, J=17.4 Hz, 1H),3.73 (s, 3H), 2.93 (dd, J=19.0 Hz, J=10.5 Hz, 1H), 2.62 (dd, J=19.0 Hz, J=3.3 Hz, 1H), 1.92-0.80 (m, 20H).

Intermediate VII was prepared from intermediate XV in a manner similar to intermediate III described in method A above. Compound 1 was then prepared from intermediate VII following the same procedures described in method A above.

### EXAMPLE 2

### Preparation of Compound 2: (8S)-7-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1,4-diethyl-2-oxo-1,4,7-triazaspiro[4.4]nonane-8-carboxamide

Compound 2 was prepared in a manner similar to method A described in Example 1.

LC/MS: 766.4 (M+H)⁺; 797.5 (M+H+MeOH)⁺; 819.5 (M+Na+MeOH)⁺.

### EXAMPLE 3

### Preparation of Compound 3: (8S)-7-((S)-2-((S)-2-cyclohexyl-2-(pyrazine-2-carboxamido)acetamido)-3,3-dimethylbutanoyl)-N-((S)-1-(cyclopropylamino)-1,2-dioxohexan-3-yl)-1,4-diethyl-2-oxo-1,4,7-triazaspiro[4.4]nonane-8-carboxamide

Compound 3 was prepared in a manner similar to method A described in Example 1.

LC/MS: 766.5 (M+H)⁺; 797.5 (M+H+MeOH)⁺; 819.5 (M+Na+MeOH)⁺.

### EXAMPLE 4

### Preparation of Compound 4: (8S)-7-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1-ethyl-4-methyl-2oxo-1,4,7-triazaspiro[4.4]nonane-8-carboxamide

Compound 4 was prepared in a manner similar to method A described in Example 1.

LC/MS: 753.2 (M+H)⁺; 784.2 (M+H+MeOH)⁺; 806.1 (M+Na+MeOH)⁺.

### EXAMPLE 5

### Preparation of Compound 5: (8S)-7-((S)-2-((S)-2-cyclohexyl-2-(pyrazine-2-carboxamido)acetamido)-3,3-dimethylbutanoyl)-N-(1-cyclopropyl-4-(cyclopropylamino)-3,4-dioxobutan-2-yl)-1-ethyl-4-methyl-2-oxo-1,4,7-triazaspiro[4.4]nonane-8-carboxamide

Compound 5 was prepared in a manner similar to method A described in Example 1.

LC/MS: 765.1 (M+H)⁺; 796.1 (M+H+MeOH)⁺; 818.0 (M+Na+MeOH)⁺.

### EXAMPLE 6

### Preparation of Compound 6: (8S)-7-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-1-ethyl-4-methyl-2-oxo-1,4,7-triazaspiro[4.4]nonane-8-carboxamide

Compound 6 was prepared in a manner similar to method A described in Example 1.

LC/MS: 817.1 (M+H)⁺; 848.1 (M+H+MeOH)⁺; 870.0 (M+Na+MeOH)⁺.

### EXAMPLE 7

### Preparation of Compound 7: tert-butyl (2S)-1-((8S)-8-(1-(2-(2-(dimethylamino)-2-oxo-1-phenylethylamino)-2-oxoethylamino)-1,2-dioxohexan-3-ylcarbamoyl)-1-ethyl-4-methyl-2-oxo-1,4,7-triazaspiro[4.4]nonan-7-yl)-3,3-dimethyl-1-oxobutan-2-ylcarbamate

Compound 7 was prepared in a manner similar to method A described in Example 1.

LC/MS: 786.0 (M+H)⁺; 817.1 (M+H+MeOH)⁺; 839.0 (M+Na+MeOH)⁺.

### EXAMPLE 8

### Preparation of Compound 8: tert-butyl (2S)-1-((5S,7a′R)-5-(1-(cycloproplamino)-1,2-dioxohexan-3-ylcarbamoyl)-1′-oxohexahydrospiro[pyrrolidine-3,3′-pyrrolo[1,2-c]imidazole]-1-yl)-3,3-dimethyl-1-oxobutan-2-ylcarbamate

Compound 8 was prepared in a manner similar to method A described in Example 1.

LC/MS: 605.1 (M+H)⁺; 637.1 (M+H+MeOH)⁺; 659.0 (M+Na+MeOH)⁺.

### EXAMPLE 9

### Preparation of Compound 9: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1′-oxohexahydrospiro[pyrrolidine-3,3′-pyrrolo[1,2-c]imidazole]-5-carboxamid

Compound 9 was prepared in a manner similar to method A described in Example 1.

LC/MS: 751.0 (M+H)⁺; 782.1 (M+H+MeOH)⁺; 804.0 (M+Na+MeOH)⁺.

### EXAMPLE 10

### Preparation of Compound 10: (5S,7a′R)-1-((S)-2-(3-(cyclohexylcarbamoyl)ureido)-3,3-dimethylbutanoyl)-N-(1-(cyclopoproplamino)-1,2-dioxohexan-3-yl)-1′-oxohexahydrospiro[pyrrolidine-3,3′-pyrrolo[1,2-c]imidazole]-5-carboxamide

Compound 10 was prepared in a manner similar to method A described in Example 1.

LC/MS: 630.7 (M+H)⁺; 661.7 (M+H+MeOH)⁺; 647.7 (M+Na+MeOH)⁺.

### EXAMPLE 11

### Preparation of Compound 11: (5S,7a′R)—N-(1-(cyclopropylamino)-1,2-dioxohexan-3-yl)-1-((S)-2-(3-cyclopropylureido)-3,3-dimethylbutanoyl)-1′-oxohexahydrospiro[pyrrolidine-3,3′-pyrrolo[1,2-c]imidazole]-5-carboxamide

Compound 11 was prepared in a manner similar to method A described in Example 1.

LC/MS: 588.7 (M+H)⁺; 619.7 (M+H+MeOH)⁺; 641.7 (M+Na+MeOH)⁺.

### EXAMPLE 12

### Preparation of Compound 12: (8S)-7-((S)-2-((S)-2-(3-tert-butylureido)-2-cyclohexylacetamido)-3,3-dimethylbutanoyl)-N-(1-(cyclopropylamino)-1,2-dioxohexan-3-yl)-1,4-diethyl-2-oxo-1,4,7-triazaspiro[4.4]nonane-8-carboxamide

Compound 12 was prepared in a manner similar to method A described in Example 1.

LC/MS: 761.1 (M+H)⁺; 792.1 (M+H+MeOH)⁺; 814.1 (M+Na+MeOH)⁺.

### EXAMPLE 13

### Preparation of Compound 13: (5S,7a′R)-2′-benzyl-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1′-oxohexahydrospiro[pyrrolidine-3,3′-pyrrolo[1,2-c]imidazole]-5-carboxamide

Compound 13 was prepared in a manner similar to method A described in Example 1.

LC/MS: 841.4 (M+H)⁺; 872.5 (M+H+MeOH)⁺; 894.3 (M+Na+MeOH)⁺.

### EXAMPLE 14

### Preparation of Compound 14: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-1′-oxohexahydrospiro[pyrrolidine-3,3′-pyrrolo[1,2-c]imidazole]-5-carboxamide

Compound 14 was prepared in a manner similar to method A described in Example 1.

LC/MS: 779.1 (M+H)⁺; 810.1 (M+H+MeOH)⁺; 832.1 (M+Na+MeOH)⁺.

### EXAMPLE 15

### Preparation of Compound 15: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-methyl-1′-oxohexahydrospiro[pyrrolidine-3,3′-pyrrolo[1,2-c]imidazole]-5-carboxamide

Compound 15 was prepared in a manner similar to method A described in Example 1.

LC/MS: 764.2 (M+H)⁺.

### EXAMPLE 16

### Preparation of Compound 16: (5S,7a′R)-1-((S)-2-((S)-2-cyclohexyl-2-(pyrazine-2-carboxamido)acetamido)-3,3-dimethylbutanoyl)-N-(1-(isopropylamino)-1,2-dioxohexan-3-yl)-2′-methyl-1′-oxohexahydrospiro[pyrrolidine-3,3′-pyrrolo[1,2-c]imidazole]-5-carboxamide

Compound 16 was prepared in a manner similar to method A described in Example 1.

LC/MS: 767.2 (M+H)⁺; 798.2 (M+H+MeOH)⁺; 820.2 (M+Na+MeOH)⁺.

### EXAMPLE 17

### Preparation of Compound 17: (5S,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenlethylamino)hexan-3-yl)-2′-methyl-1′-oxohexahydrospiro[pyrrolidine-3,3′-pyrrolo[1,2-c]imidazole]-5-carboxamide

Compound 17 was prepared in a manner similar to method A described in Example 1.

LC/MS: 829.2 (M+H)⁺; 860.2 (M+H+MeOH)⁺; 882.2 (M+Na+MeOH)⁺.

### EXAMPLE 18

### Preparation of Compound 18: (5S,7a′S)-1-((S)-2-((S)-2-cyclohexyl-2-(pyrazine-2-carboxamido)acetamido)-3,3-dimethylbutanoyl)-N-(1-(2-(2-(dimethylamino)-2oxo-1-phenylethylamino)-2-oxoethylamino)-1,2-dioxohexan-3-yl)-2′-methyl-1′-oxohexahydrospiro[pyrrolidine-3,3′-pyrrolo[1,2-c]imidazole]-5-carboxamide

Compound 18 was prepared in a manner similar to method A described in Example 1.

LC/MS: 943.5 (M+H)⁺; 974.5 (M+H+MeOH)⁺; 996.5 (M+Na+MeOH)⁺.

### EXAMPLE 19

### Preparation of Compound 19: (5S,6′R,7a′R)-6′-(benzyloxy)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-methyl-1′-oxohexahydrospiro[pyrrolidine-3,3′-pyrrolo[1,2-c]imidazole]-5carboxamide

Compound 19 was prepared in a manner similar to method B described in Example 1.

LC/MS: 949.1 (M+H)⁺; 980.1 (M+H+MeOH)⁺.

### EXAMPLE 20

### Preparation of Compound 20: (5S,6′R,7a′R)-6′-(allyloxy)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-ethyl-1′-oxohexahydrospiro[pyrrolidine-3,3′-pyrrolo[1,2-c]imidazole]-5-carboxamide

Compound 20 was prepared in a manner similar to method B described in Example 1.

LC/MS: 899.4 (M+H)⁺; 930.5 (M+H+MeOH)⁺; 952.4 (M+Na+MeOH)⁺.

### EXAMPLE 21

### Preparation of Compound 21: (5S,7a′R)-1-((S)-2-((S)-2-cyclohexyl-2-(pyrazine-2-carboxamido)acetamido)-3,3-dimethylbutanoyl)-N-(1-(cyclopropylamino)-6-methyl-1,2-dioxoheptan-3-yl)-2′-ethyl-1′-oxohexahydrospiro[pyrrolidine-3,3′-pyrrolo[1,2-c]imidazole]-5-carboxamide

Compound 21 was prepared in a manner similar to method B described in Example 1.

LC/MS: 807.1 (M+H)⁺; 838.1 (M+H+MeOH)⁺; 860.1 (M+Na+MeOH)⁺.

### EXAMPLE 22

### Preparation of Compound 22: (5S,7a′R)-1-((S)-2-((S)-2-cyclohexyl-2-(pyrazine-2-carboxamido)acetamido)-3,3-dimethylbutanoyl)-N-(1-(cyclopropylamino)-5-methyl-1,2-dioxohexan-3-yl)-2′-ethyl-1′-oxohexahydrospiro [pyrrolidine-3,3′-pyrrolo[1,2-c]imidazole]-5-carboxamide

Compound 22 was prepared in a manner similar to method B described in Example 1.

LC/MS: 793.1 (M+H)⁺; 824.1 (M+H+MeOH)⁺; 846.0 (M+Na+MeOH)⁺.

### EXAMPLE 23

### Preparation of Compound 23: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxoheptan-3-yl)-2′-ethyl-1′-oxohexahydrospiro[pyrrolidine-3,3′-pyrrolo[1,2-c]imidazole]-5-carboxamide

Compound 23 was prepared in a manner similar to method B described in Example 1.

LC/MS: 793.1 (M+H)⁺; 824.2 (M+H+MeOH)⁺; 846.2 (M+Na+MeOH)⁺.

### EXAMPLE 24

### Preparation of Compound 24: (5S,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-ethyl-1′-oxohexahydrospiro[pyrrolidine-3,3′-pyrrolo[1,2-c]imidazole]-5-carboxamide

Compound 24 was prepared in a manner similar to method B described in Example 1.

LC/MS: 843.4 (M+H)⁺; 874.4 (M+H+MeOH)⁺; 896.3 (M+Na+MeOH)⁺.

### EXAMPLE 25

### Preparation of Compound 25: (5S,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-ethyl-1′-oxohexahydrospiro[pyrrolidine-3,3′-pyrrolo[1,2-c]imidazole]-5-carboxamide

Compound 25 was prepared in a manner similar to method B described in Example 1.

LC/MS: 843.5 (M+H)⁺; 874.5 (M+H+MeOH)⁺; 896.5 (M+Na+MeOH)⁺.

### EXAMPLE 26

### Preparation of Compound 26: (5S,6′R,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-ethyl-6′-hydroxy-1′-oxohexahydrospiro[pyrrolidine-3,3′-pyrrolo[1,2-c]imidazole]-5-carboxamide

Compound 26 was prepared in a manner similar to method B described in Example 1.

LC/MS: 859.1 (M+H)⁺; 890.5 (M+H+MeOH)⁺.

### EXAMPLE 27

### Preparation of Compound 27: (5S,6′R,7a′R)-1-((S)-2-((S)-2-cyclohexyl-2(pyrazine-2 2-carboxamido)acetamido)-3,3-dimethylbutanoyl)-N-(1,2-dioxo-1-((S)-1-phenylethylamino)hexan-3-yl)-2′-ethyl-6′-(3-methylbutanamido)-1′-oxohexahydrospiro[pyrrolidine-3,3′-pyrrolo[1,2-c]imidazole]-5-carboxamide

Compound 27 was prepared in a manner similar to method B described in Example 1.

LC/MS: 942.5 (M+H)⁺; 973.5 (M+H+MeOH)⁺; 995.5 (M+Na+MeOH)⁺.

### EXAMPLE 28

### Preparation of Compound 28: (5S,7a′R)-1-((S)-2-((S)-2-cyclohexyl-2-(pyrozine-2-carboxamido)acetamido)-3,3-dimethylbutanoyl)-N-(1,2-dioxo-1-((S)-1-phenylethlamino)hexan-3-yl)-2′-isobutyl-1′-oxohexahydrospiro[pyrrolidine-3,3′-pyrrolo [1,2-c]imidazole]-5-carboxamide

Compound 28 was prepared in a manner similar to method B described in Example 1.

LC/MS: 871.5 (M+H)⁺; 902.5 (M+H+MeOH)⁺; 924.5 (M+Na+MeOH)⁺.

### EXAMPLE 29

### Preparation of Compound 29: (5S,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-2′-(3-methoxybenzyl)-1′-oxohexahydrospiro[pyrrolidine-3,3′-pyrrolo[1,2-c]imidazole]-5-carboxamide

Compound 29 was prepared in a manner similar to method B described in Example 1.

LC/MS: 935.5 (M+H); 966.5 (M+H+MeOH)⁺; 988.5 (M+Na+MeOH)⁺.

### EXAMPLE 30

### Preparation of Compound 30: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-(3-methoxybenzyl)-1′-oxohexahydrospiro[pyrrolidine-3,3′-pyrrolo[1,2-c]imidazole]-5-carboxamide

Compound 30 was prepared in a manner similar to method B described in Example 1.

LC/MS: 871.4 (M+H)⁺; 902.4 (M+H+MeOH)⁺; 924.4 (M+Na+MeOH)⁺.

### EXAMPLE 31

### Preparation of Compound 31: (5S,7a′R)-2′-allyl-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-1′-oxohexahydrospiro[pyrrolidine-3,3′-pyrrolo[1,2-c]imidazole]-5-carboxamide

Compound 31 was prepared in a manner similar to method B described in Example 1.

LC/MS: 855.4 (M+H)⁺.

### EXAMPLE 32

### Preparation of Compound 32: (5S,7a′S)-2′-allyl-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1′-oxohexahydrospiro[pyrrolidine-3,3′-pyrrolo[1,2-c]imidazole]-5-carboxamid

Compound 32 was prepared in a manner similar to method B described in Example 1.

LC/MS: 791.5 (M+H)⁺.

### EXAMPLE 33

### Preparation of Compound 33: (5S,7a′R)-1-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-1′-oxo-2′-(prop-2-ynyl)hexahydrospiro[pyrrolidine-3,3′-pyrrolo[1,2-c]imidazole]-5-carboxamide

Compound 33 was prepared in a manner similar to method B described in Example 1.

LC/MS: 853.4 (M+H)⁺; 884.4 (M+H+MeOH)⁺; 906.3 (M+Na+MeOH)⁺.

### EXAMPLE 34

### Preparation of Compound 34: (5S,7a′R)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-1′-oxo-2′-(prop-2-ynyl)hexahydrospiro[pyrrolidine-3,3′-pyrrolo[1,2-c]imidazole]-5-carboxamide

Compound 34 was prepared in a manner similar to method B described in Example 1.

LC/MS: 789.5 (M+H)⁺; 820.2 (M+H+MeOH)⁺; 842.2 (M+Na+MeOH)⁺.

### EXAMPLE 35

### Preparation of Compound 35: (8S)-7-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2,4-dioxo-1,3,7-triazaspiro[4.4]nonane-8-carboxamide

Compound 35 was prepared by the method illustrated in Scheme 7 below.

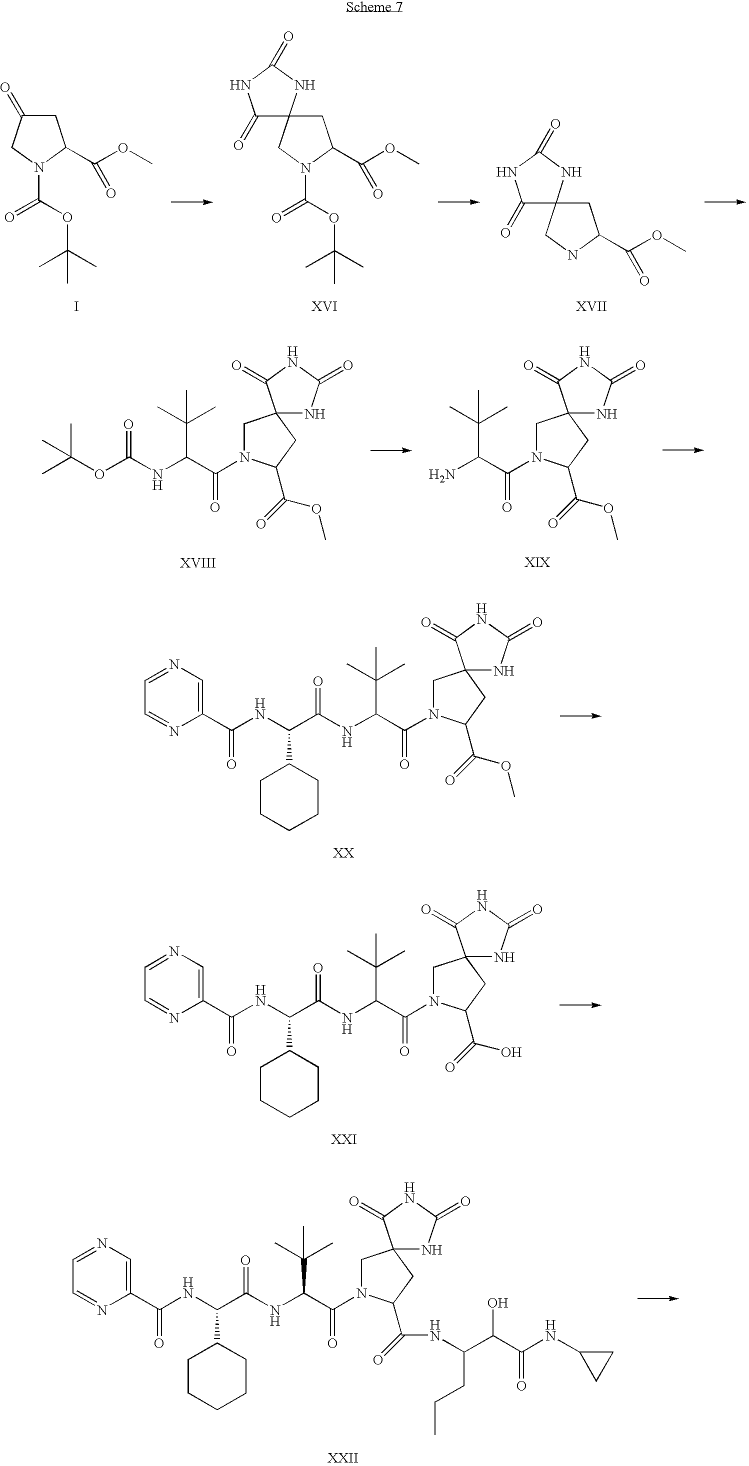

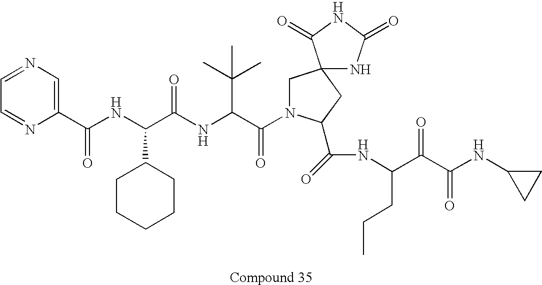

Compound I (2.9 g, 12 mmol) in methanol was added to a solution of ammonium carbonate (5.76 g, 60 mmol) and potassium cyanide (1.56 g, 24 mmol) in methanol/water (1:1) (60 mL). After the mixture was heated at 55-60° C. for 24 hours, the solvent was removed under vacuum. The residue thus obtained was diluted with water (20 mL), and the mixture was extracted with ethyl acetate (80 mL). The organic layer was collected, dried over anhydrous MgSO₄, concentrated, and purified by silica gel chromatography to afford 2.6 g (yield: 70%) of intermediate XVI. LC/MS: 214.0 (M+H)⁺.

To a solution of intermediate XVI (3.1 g, 10 mmol) in CH₂Cl₂ (150 mL) was added a solution of 4.0 N HCl in dioxane (15 mL). The reaction mixture was stirred at room temperature for 3 hours. The resulting mixture was concentrated to give a quantitative yield of crude intermediate XVII, which was used in the next step without further purification.

N-methyl morphorline (3 mL) was added to a solution of intermediate XVII (2.1 g, 10.0 mmol), EDC (2.9 g, 15.0 mmol), Bu^{t}OH (1.4 g, 10.0 mmol) and 2-tert-butoxycarbonylamino-3,3-dimethyl-butyric acid (2.3 g, 34.4 mmol) in CH₂Cl₂ (150 mL) at room temperature. After the reaction mixture was stirred at the same temperature overnight, it was quenched with water. The mixture was then extracted with CH₂Cl₂. The organic layer was collected, dried with anhydrous MgSO₄, concentrated, and purified by silica gel chromatography to afford 3.2 g (yield: 75%) of compound XVIII. LC/MS: 371.0 (M+H)^{+.}

To a solution of intermediate XVIII (2.1 g, 5.0 mmol) in CH₂Cl₂ (100 mL) was added a solution of 4.0 N HCl in dioxane (15 mL). The reaction mixture was stirred at room temperature for 3 hours. The mixture was concentrated to afford a quantitative yield of the crude intermediate XIX, which was used in the next step without further purification.

N-methyl morphorline (2.0 mL) and DMF (5 mL) were added to a solution of intermediate XIX (1.6 g, 5.0 mmol), EDC (1.5 g, 7.5 mmol), Bu^{t}OH (0.68 g, 5.0 mmol), and cyclohexyl-[(pyrazine-2-carbonyl)-amino]-acetic acid (1.3 g, 5.0 mmol) in CH₂Cl₂ (100 mL) at room temperature. After the reaction mixture was stirred at the same temperature overnight, it was quenched with water. The mixture was then extracted with CH₂Cl₂. The organic layer was collected, dried with anhydrous MgSO₄, concentrated, and purified by silica gel chromatography to obtain 1.9 g (yield: 66%) of intermediate XX. LC/MS: 572.1 (M+H)⁺; 594.0 (M+Na)⁺.

To a solution of compound XX (557 mg, 1.0 mmol) in THF (30 mL) was added a solution of 0.5 M LiOH (10 mL). After the reaction mixture was stirred at room temperature for 3 hours, it was neutralized to pH 5˜6 with 1N HCl. The mixture was then extracted with CH₂Cl₂ (60 mL). The organic layer was collected and concentrated to afford a crude intermediate XXI, which was used in the next step without further purification.

N-methyl morphorline (1 mL) was added to a solution of intermediate XXI (278.5 mg, 0.5 mmol), EDC (148.3 mg, 0.75 mmol), 1-hydroxybenzotriazole (HOBₜ, 67.5 mg, 0.5 mmol) and 3-amino-2-hydroxy-hexanoic acid cyclopropylamide (intermediate IX, 93.0 mg, 0.5 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified by silica gel chromatography to obtain 266 mg (yield: 73%) of intermediate XXII. LC/MS: 726.8 (M+H)⁺; 748.2 (M+Na)⁺.

To a solution of intermediate XXII (145 mg, 0.2 mmol) in CH₂Cl₂ (30 mL) was added Dess-Martin reagent (145 mg, 0.36 mmol) at room temperature. After the reaction mixture was stirred at the same temperature for 3 hours, it was quenched with an IN NaOH (5 mL) aqueous solution. The mixture was then extracted with CH₂Cl₂ (50 mL). The organic layer was collected, concentrated, and purified by silica gel chromatography to afford 130 mg (yield: 90%) of compound 35. LC/MS: 725.0 (M+H)⁺. ¹H-NMR (CDCl₃): δ 9.38 (s, 1H), 8.78 (s, 1H), 8.56 (s, 1H), 8.36-7.16 (m, 5H), 5.61-3.85 (m, 5H), 2.98-2.28 (m, 3H), 2.07-0.75 (m, 33H).

### EXAMPLE 36

### Preparation of Compound 36: (1R, 2S)-1-((5S, 7a′S)-1-((S)-2-((S)-2-cyclohexyl-2-(pyrazine-2-carboxamido)acetamido)-3,3-dimethylbutanoyl)-2′-ethyl-1′-oxohexahydrospiro[pyrrolidine-3,3′-pyrrolo[1,2-c]imidazole]-5-ylcarboxamido)-2-vinylcyclopropanecarboxylic acid

Compound 36 was prepared by the method illustrated in Scheme 8 below.

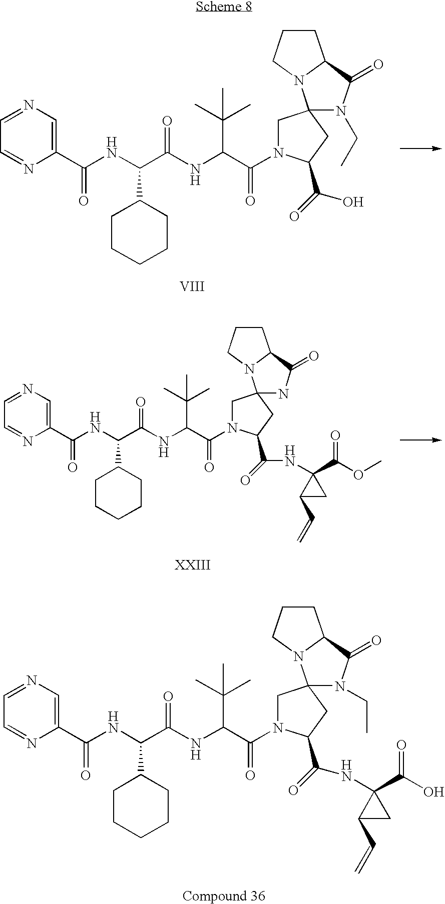

N-methyl morphorline (1 mL) was added to a solution of intermediate VIII prepared in Example 1 (183.5 mg, 0.3 mmol), [O-(7-azabenzotriazo-1-yl)-1,1,3,3-tetramethyluronium hexafluorophosphate] (HATU, 228.0 mg, 0.60 mmol), and 1-amino-2-vinyl-cyclopropanecarboxylic acid methyl ester (63.5 mg, 0.45 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified by silica gel column chromatography to afford 150 mg (68% yield) of intermediate XXIII.

To a solution of intermediate XXIII (220.2 mg, 0.3 mmol) in THF/methanol (3:1) (40 mL) was added a solution of 0.5 M LiOH (10 mL). The reaction mixture was stirred at room temperature for 3 hours. After the mixture was neutralized to pH of 5-6 with an 1N HCl aqueous solution, it was extracted with CH₂Cl₂ (100 mL). The organic layer was collected and concentrated to afford a crude product, which was purified by silica gel column chromatography to obtain compound 36 in a 70% yield. LC/MS: 721.3 (M+H)⁺.

### EXAMPLE 37

### Preparation of Compound 37: (5S, 7a′S)-1-((S)-2-((S)-2-cyclohexyl-2-(pyrazine-2-carboxamido)acetamido)-3,3-dimethylbutanoyl)-2′-ethyl-1′-oxo-N-((1R, 2S)-1-(phenylsulfonylcarbamoyl)-2-vinylcyclopropyl)hexahydrospiro[pyrrolidine-3,3′-pyrrolo[1,2-c]imidazole]-5-carboxamide

After a solution of compound 36 (144 mg, 0.2 mmol), N,N′-diisopropylethylamine (DIPEA, 155.2 mg, 1.2 mmol), and HATU (456.0 mg, 1.2 mmol) in CH₂Cl₂ (60 mL) was stirred at room temperature for 1.5 hours, 4-(dimethylamino)-pyridine (DMAP, 122.2 mg, 1.0 mmol) and benzenesulfonamide (62.9 mg, 0.4 mmol) were added at the same temperature. The reaction mixture was stirred for another 15 minutes. 1,8-Diazabicyclo(5.4.0)undec-7-ene (DBU, 152.2 mg, 1.0 mmol) was then added slowly at room temperature. After stirred at that temperature for overnight, the reaction mixture was quenched with water, and was extracted with CH₂Cl₂ (60 mL). The organic layer was collected, dried, concentrated, and purified by silica gel chromatography to obtain 99.8 mg (yield: 58%) of compound 37. LC/MS: 861.4 (M+H)⁺. ¹H-NMR (CDCl₃) δ 9.40 (d, J=8.1 Hz, 1H), 8.75-7.16 (m, 10 H), 5.19-2.71 (m, 8H), 2.51-0.99 (m, 37H).

### EXAMPLE 38

### Preparation of Compound 38: (8S)-7-((S)-2-((S)-2-cyclohexyl-2-(pyrazine-2-carboxamido)acetamido)-3,3-dimethylbutanoyl)-N-(1,2-dioxo-1-((S)-1-phenylethylamino)hexan-3-yl)-3-oxo-1-thia-4,7-diazaspiro[4.4]nonane-8-carboxamide

Compound 38 was prepared by the method illustrated in Scheme 9 below.

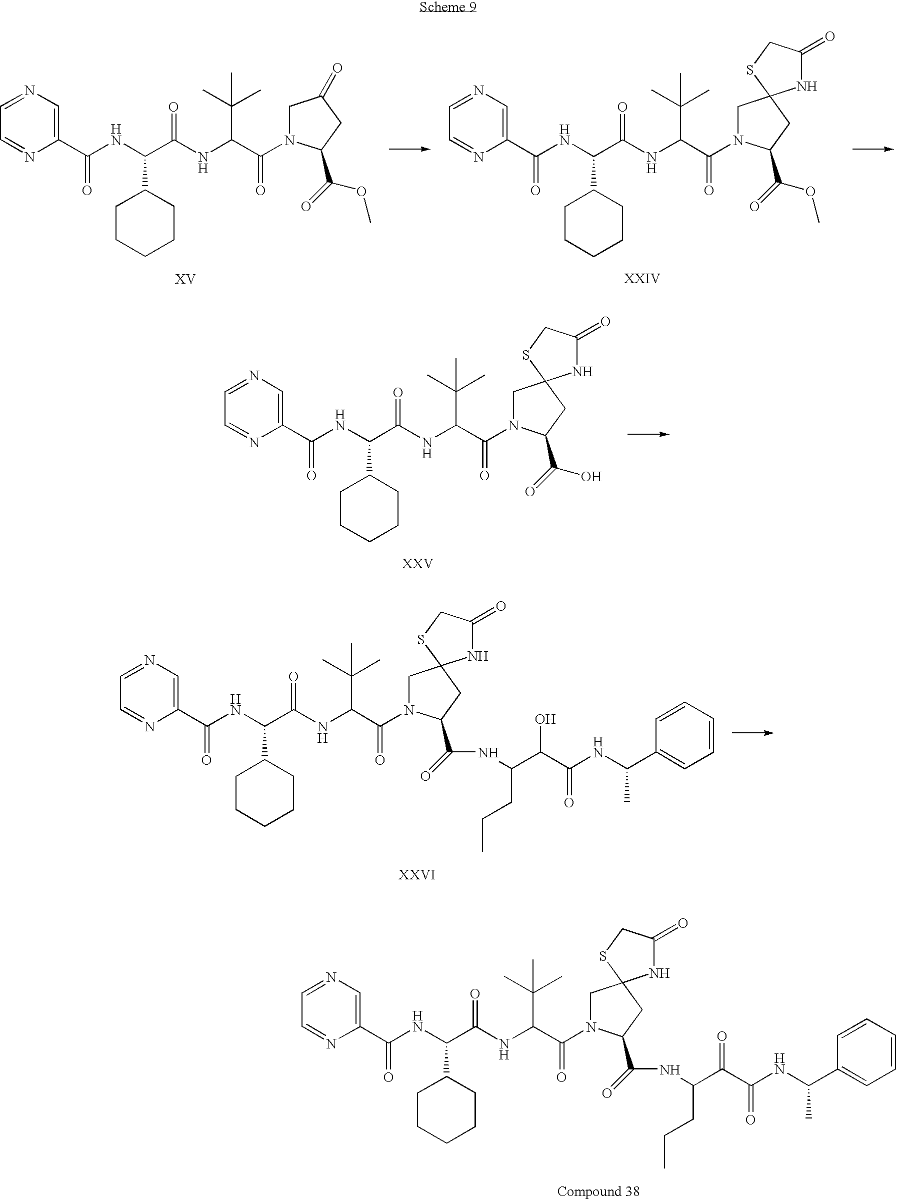

A solution of intermediate XV prepared in Example 1 (250.8 mg, 0.5 mmol), ammonium acetate (77.0 mg, 1.0 mmol,) and thioglycolic acid (48 mg, 0.55 mmol) in benzene (40 mL) was refluxed for 10 hours. The reaction solution was then quenched with a saturated NH₄Cl aqueous solution and extracted with CH₂Cl₂ (100 mL). The organic layer was collected, concentrated, and purified by silica gel chromatography to afford 229.8 mg (yield: 80%) of intermediate XXIV. LC/MS: 575.2 (M+H)⁺.

To a solution of intermediate XXIV (143.7 mg, 0.25 mmol) in THF (30 mL) was added an aqueous solution of 0.5 M LiOH (10 mL). The reaction mixture was stirred at room temperature for 3 hours. After the mixture was neutralized to pH of 5˜6 with an 1N HCl aqueous solution, it was extracted with CH₂Cl₂ (60 mL). The organic layer was collected and concentrated to afford a crude intermediate XXV, which was used in the next step without further purification. LC/MS: 561.2 (M+H)⁺.

N-methyl morphorline (0.5 mL) was added to a solution of intermediate XXV (112.1 mg, 0.2 mmol), EDC (59.3 mg, 0.3 mmol), HOBt (27.0 mg, 0.2 mmol), and 3-amino-2-hydroxy-hexanoic acid cyclopropylamide (intermediate IX, 37.2 mg, 0.2 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified by silica gel chromatography to afford 119.0 mg (yield: 75%) of intermediate XXVI. LC/MS: 793.3 (M+H)⁺; 815.3 (M+Na)⁺.

To a solution of intermediate XXVI (103.1 mg, 0.13 mmol) in CH₂Cl₂ (30 mL) was added Dess-Martin reagent (52.4 mg, 0.36 mmol) at room temperature. After the reaction mixture was stirred at the same temperature for 3 hours, it was quenched with an IN NaOH aqueous solution (3 mL). The mixture was then extracted with CH₂Cl₂ (50 mL), concentrated under vacuum, and purified by silica gel chromatography to afford 84.2 mg (yield: 82%) of compound 38. LC/MS: 791.4 (M+H)+. ¹H-NMR (CDCl₃): δ 9.39 (d, J=6.9 Hz, 1H), 8.74 (s, 1H), 8.53 (s, 1H), 8.31-8.20 (m, 1H), 7.43-6.78 (m, 8H), 5.42-4.93 (m, 2H), 4.66-4.42 (m, 2H), 4.16-4.02 (m, 2H), 3.74-3.40 3.05 (m, 1H), 2.60-2.26 (m, 2H), 1.92-0.74 (m, 30H).

### EXAMPLE 39

### Preparation of Compound 39: (8S)-7-((S)-2-((S)-2-cyclohexyl-2-(pyrazine-2-carboxamido)acetamido)-3,3-dimethylbutanoyl)-4-(cyclopropylmethyl)-N-(1,2-dioxo-1-((S)-1-phenylethylamino)hexan-3-yl)-3-oxo-1-thia-4,7-diazaspiro[4.4]nonane-8-carboxamide

Compound 39 was prepared by the method illustrated in Scheme 10 below.

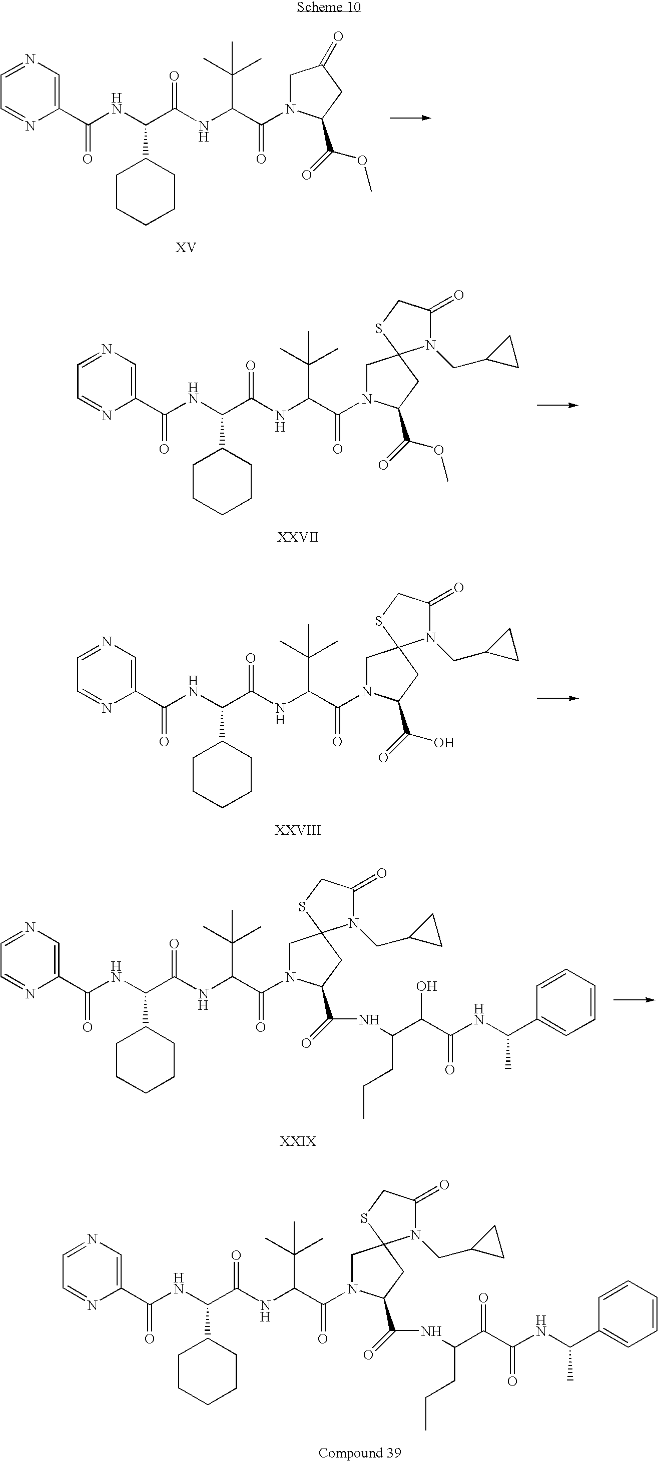

A solution of intermediate XV (501.6 mg, 1.0 mmol), cyclopropanemethylamine (85.3 mg, 1.2 mmol), and thioglycolic acid (276.3 mg, 3.0 mmol) in THF were stirred at 0° C. for 50 minutes. After DCC (247.6 mg, 1.2 mmol) was added at the same temperature, the reaction mixture was warmed to room temperature and stirred for additional 5 hours. The mixture was then quenched with a saturated NH₄Cl aqueous solution and extracted with CH₂Cl₂ (100 mL). The organic layer was collected, concentrated under vacuum, and purified by silica gel chromatography to afford 376.8 mg (yield: 60%) of intermediate XXVII. LC/MS: 629.6 (M+H)⁺.

To a solution of intermediate XXVII (314.4 mg, 0.5 mmol) in THF (30 mL) was added an aqueous solution of 0.5 M LiOH (10 mL). The reaction mixture was stirred at room temperature for 3 hours. After the mixture was neutralized to pH of 5-6 with an 1N HCl aqueous solution, it was extracted with CH₂Cl₂ (60 mL). The organic layer was collected and concentrated to afford a crude intermediate XXVIII, which was used in the next step without further purification. LC/MS: 615.5 (M+H)⁺.

N-methyl morphorline (1 mL) was added to a solution of intermediate XXVIII (307.4 mg, 0.5 mmol), EDC (148.3 mg, 0.75 mmol), HOBt (67.5 mg, 0.5 mmol) and 3-amino-2-hydroxy-hexanoic acid cyclopropylamide (intermediate IX, 93.0 mg, 0.5 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified by silica gel chromatography to afford 330.4 mg (yield: 78%) of intermediate XXIX. LC/MS: 847.5 (M+H)⁺.

To a solution of intermediate XXIX (169.1 mg, 0.2 mmol) in CH₂Cl₂ (30 mL) was added Dess-Martin reagent (145 mg, 0.36 mmol) at room temperature. After the reaction mixture was stirred at the same temperature for 3 hours, it was quenched with an 1N NaOH aqueous solution (5 mL). The mixture was then extracted with CH₂Cl₂ (50 mL). The organic layer was collected, concentrated, and purified by silica gel chromatography to afford 126.8 mg (yield: 75%) of compound 39. LC/MS: 845.4 (M+H)⁺. ¹H-NMR (CDCl₃) δ 9.46-9.37 (m, 1H), 8.73 (s, 1H), 8.54 (s, 1H), 8.36-8.20 (m, 1H), 7.63-6.52 (m, 8H), 5.54-4.35 (m, 6H), 4.19-2.84 (m, 9H), 2.38-2.20 (m, 1H), 1.92-0.81 (m, 28H), 0.58-0.43 (m, 2H), 0.42-0.24 (m, 2H).

### EXAMPLE 40

### Preparation of Compound 40: (5′S)-1′-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-4-oxospiro[chroman-2,3′-pyrrolidine]-5′-carboxamide

Compound 40 was prepared by the method illustrated in Scheme 11 below.

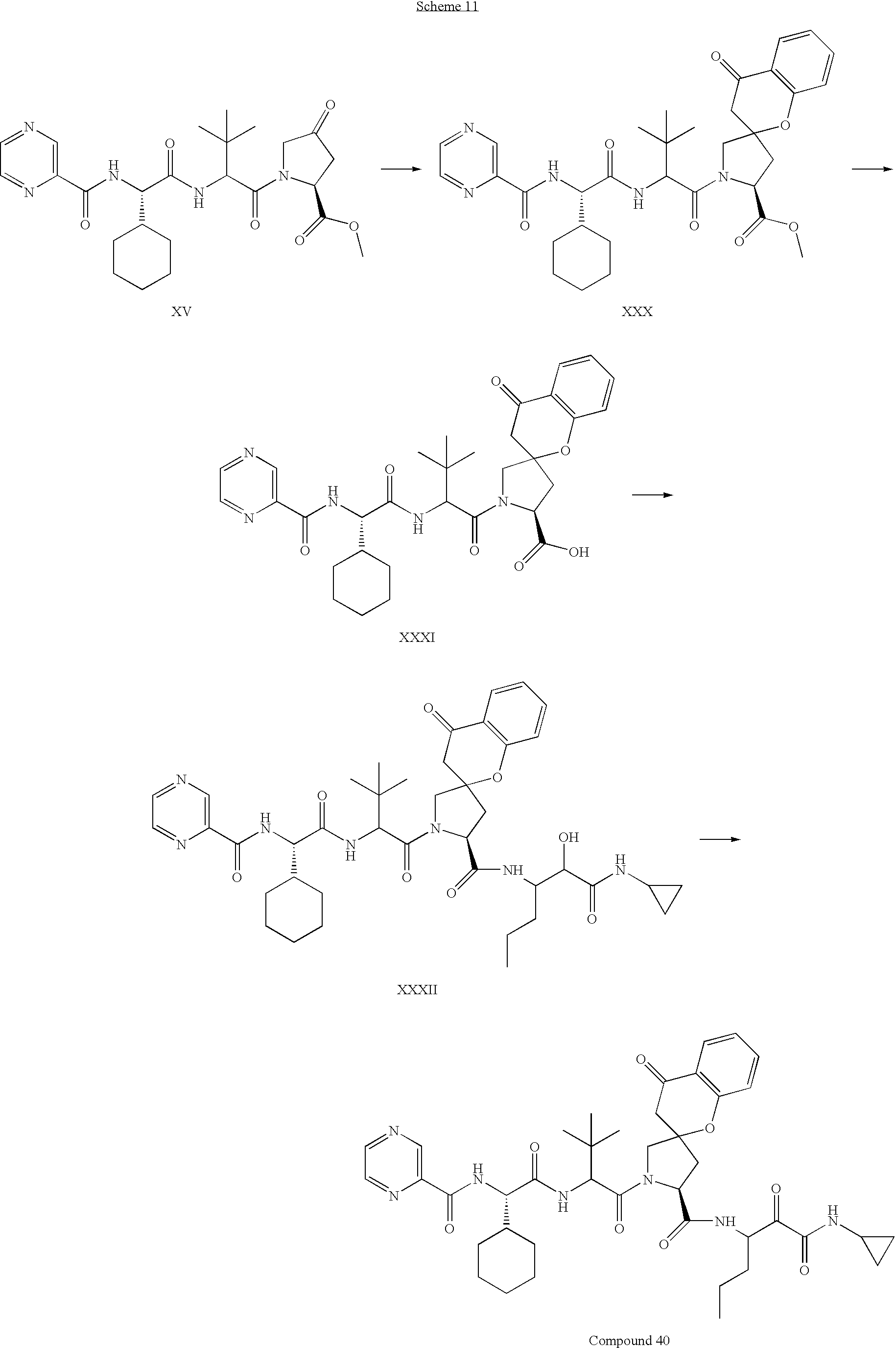

A solution of intermediate XV prepared in Example 1 (501.6 mg, 1.0 mmol), pyrrolidine (106.5 mg, 1.5 mmol) and 2-hydroxyacetophenone (204.3 mg, 1.5 mmol) in methanol (40 mL) was refluxed for 12 hours. The reaction mixture was concentrated and purified by silica gel chromatography to afford 340.8 mg (yield: 55%) of intermediate XXX. LC/MS: 620.7 (M+H)⁺. ¹H-NMR (CDCl₃): δ 9.38 (s, 1H), 8.76 (brs, 1H), 8.55 (brs, 1H), 8.26 (d, J=9 Hz, 1H), 7.90 (d, J=7.8 Hz, 1H), 7.55-7.45 (m, 1H), 7.07 (dd, J=15.2 Hz, J=7.8 Hz, 1H), 6.89-6.79 (m, 1H), 6.61-6.53 (m, 1H), 5.21-4.3 (m, 3H), 4.15-3.65 (m, 4H), 3.13-2.59 (m, 2H), 2.10-1.47 (m, 8H), 1.35-0.80 (m, 15H).

To a solution of intermediate XXX (309.9 mg, 0.5 mmol) in THF (30 mL) was added an aqueous solution of 0.5 M LiOH (10 mL). The reaction mixture was stirred at room temperature for 3 hours. After the mixture was neutralized to pH of 5˜6 with an 1N HCl aqueous solution, it was extracted with CH₂Cl₂ (60 mL). The organic layer was collected and concentrated to afford a crude intermediate XXXI, which was used in the next step without further purification.

N-methyl morphorline (1 mL) was added to a solution of intermediate XXXI (302.9 mg, 0.5 mmol), EDC (148.3 mg, 0.75 mmol), HOBt (67.5 mg, 0.5 mmol) and 3-amino-2-hydroxy-hexanoic acid cyclopropylamide (intermediate IX, 93.0 mg, 0.5 mmol) in CH₂Cl₂ (30 mL) at room temperature. After the reaction mixture was stirred at the same temperature overnight, it was quenched with water. The mixture was then extracted with CH₂Cl₂ (60 mL). The organic layer was collected, dried, concentrated, and purified by silica gel chromatography to afford 301.8 mg (yield: 78%) of intermediate XXXII. LC/MS: 774.7 (M+H)⁺; 796.7 (M+Na)⁺. ¹H-NMR (CDCl₃): δ 9.52-9.29 (m, 1H), 8.81-8.75 (m, 1H), 8.60-8.52 (m, 1H), 8.33-8.20 (m, 1H), 7.93-7.84 (m, 1H), 7.54-7.25 (m, 2H), 7.11-6.78 (m, 4H), 5.30-4.96 (m, 1H), 4.86-4.28 (m, 3H), 4.27-3.92 (m, 3H), 3.78-3.61 (m, 1H), 3.15-2.52 (m, 3H), 2.33-1.97 (m, 2H), 1.96-1.55 (m, 10H), 1.48-0.40 (m, 21H).

To a solution of intermediate XXXII (154.8 mg, 0.2 mmol) in CH₂Cl₂ (30 mL) was added Dess-Martin reagent (145 mg, 0.36 mmol) at room temperature. After the reaction mixture was stirred at the same temperature for 3 hours, it was quenched with an 1N NaOH aqueous solution (5 mL). The mixture was then extracted with CH₂Cl₂ (50 mL). The organic layer was collected, concentrated under vacuum, and purified by silica gel chromatography to afford 111.2 mg (yield: 72%) of compound 40. LC/MS: 772.7 (M+H)^{+.} ¹H-NMR (CDCl₃): δ 9.43-9.39 (m, 1H), 8.76-8.74 (m, 1H), 8.55-8.53 (m, 1H), 8.28-8.23 (m, 1H), 7.91-7.84 (m, 1H), 7.53-7.35 (m, 2H), 7.18-6.96 (m, 2H), 6.91-6.72 (m, 2H), 5.46-5.23 (m, 1H), 4.91-4.78 (m, 1H), 4.65-4.48 (m, ₂H), 4.43-4.25 (m, 1H), 4.05 (s, 1H), 3.75-3.47 (m, 1H), 3.22-2.72 (m, 4H), 2.61-2.30 (m, 1H), 2.18-1.49 (m, 9H), 1.45-0.55 (m, 21H).

### EXAMPLE 41

### Preparation of Compound 41: (5S, 6′S, 7a′S)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-1′-oxo-6′-phenoxyhexahydrospiro[pyrrolidine-3,3′-pyrrolo[1,2-c]imidazole]-5-carboxamide

Compound 41 was prepared in a manner similar to method A described in Example 1.

LC/MS: 870.5 (M+H)⁺, 902.5 (M+H+MeOH)⁺, 924.5 (M+Na+MeOH)⁺.

### EXAMPLE 42

### Preparation of Compound 42: (5S, 6′S, 7a′S)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-6′-(3-fluorophenoxy)-1′-oxohexahydrospiro[pyrrolidine-3,3′-pyrrolo[1,2-c]imidazole]-5-carboxamide

Compound 42 was prepared in a manner similar to method A described in Example 1.

LC/MS: 888.5 (M+H)⁺, 920.5 (M+H+MeOH)+, 942.5 (M+Na+MeOH)⁺.

### EXAMPLE 43

### Preparation of Compound 43: (5S, 6′S, 7a′S)-6′-(3-chlorophenoxy)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-1′-oxohexahydrospiro[pyrrolidine-3,3′-pyrrolo[1,2-c]imidazole]-5-carboxamide

Compound 43 was prepared in a manner similar to method A described in Example 1.

LC/MS: 904.1 (M+H)⁺, 936.1 (M+H+MeOH)+, 958.1 (M+Na+MeOH)+.

### EXAMPLE 44

### Preparation of Compound 44: (5S, 6′S, 7a′S)-6′-(benzo[d][1,3]dioxol-5-yloxy)-1-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-2′-ethyl-1′-oxohexahydrospiro[pyrrolidine-3,3′-pyrrolo[1,2-c]imidazole]-5-carboxamide

Compound 44 was prepared in a manner similar to method A described in Example 1.

LC/MS: 913.9 (M+H)⁺, 945.9 (M+H+MeOH)⁺, 967.8 (M+Na+MeOH)⁺.

### EXAMPLE 45

### Preparation of Compound 45: (5S, 6′S, 7a′S)-6′-(3-chlorophenoxy)-1-((S)-2-((S)-2-cyclohexyl-2-(1-methyl-1H-pyrrole-2-carboxamido)acetamido)-3,3-dimethylbutanoyl)-N-(1-(cyclopropylamino)-1,2-dioxohexan-3-yl)-2′-ethyl-1′-oxohexahydrospiro[pyrrolidine-3,3′-pyrrolo[1,2-c]imidazole]-5-carboxamide

Compound 45 was prepared in a manner similar to method A described in Example 1.

LC/MS: 905.5 (M+H)⁺, 937.5 (M+H+MeOH)⁺, 959.5 (M+Na+MeOH)⁺.

### EXAMPLE 46

### Preparation of Compound 46: N-((1S)-2-((2S)-1-((5S, 6′S, 7a′S)-6′-(3-chlorophenoxy)-5-(1-(cyclopropylamino)-1,2-dioxohexan-3-ylcarbamoyl)-2′-ethyl-1′-oxohexahydrospiro[pyrrolidine-3,3′-pyrrolo[1,2-c]imidazole]-1-yl)-3,3-dimethyl-1-oxobutan-2-ylamino)-1-cyclohexyl-2-oxoethyl)-4-methylthiazole-5-carboxamide

Compound 46 was prepared in a manner similar to method A described in Example 1.

LC/MS: 923.4 (M+H)⁺, 945.4 (M+Na)⁺, 977.4 (M+Na+MeOH)⁺.

### EXAMPLE 47

### Preparation of Compound 47: (5′S)-1′-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-3-methyl-4-oxo-3,4-dihydrospiro[benzo[e][1,3]thiazine-2,3′-pyrrolidine]-5-carboxamide

Compound 47 was prepared in a manner similar to the method described in Example 39.

LC/MS: 803.4 (M+H)⁺, 935.4 (M+H+MeOH)⁺, 957.4 (M+Na+MeOH)⁺.

### EXAMPLE 48

### Preparation of Compound 48: (5′S)-1′-((S)-2-((S)-2-cyclohexyl-2-(pyrazine-2-carboxamido)acetamido)-3,3-dimethylbutanoyl)-N-(1-(cyclopropylamino)-1,2-dioxohexan-3-yl)-6-fluoro-3-methyl-4-oxo-3,4-dihydrospiro [benzo [e][1,3]thiazine-2,3′-pyrrolidine]-5′-carboxamide

Compound 48 was prepared in a manner similar to the method described in Example 39.

LC/MS: 820.9 (M+H)⁺, 842.9 (M+Na)⁺, 874.9 (M+Na+MeOH)⁺.

### EXAMPLE 49

### Inhibition of NS3/4 A Protease

### Protein Expression and Purification

A plasmid containing N-terminal His₆₋tagged-NS4A ₍₂₁₋₃₂₎-GSGS-NS3₍₃₋₁₈₁₎ was transformed into E. coli strain BL21 (DE3) pLysS (Novagen) for protein over-expression. Single colony of transformed BL21 (DE3) pLysS was cultured in 200 mL of Lauria-Bertani (LB) medium with Kanamycin and Chloramphenicol at 37° C. overnight. The bacterial culture was transferred into 6 L LB medium (Difco) containing antibiotics and incubated with shaking at 22° C. After the absorbance at 600 nm reached 0.6, the culture was induced with 1 mM isopropyl-1-thio-β-D-galactopyranoside (IPTG) at 22° C. for 5 hours. The culture was subsequently harvested by centrifugation (6,000 xg for 15 minutes at 4° C.). Cell pellets were resuspended in 150 mL buffer A (50 mM HEPES, pH 7.4, 0.3 M NaCl, 0.1% (w/v) CHAPS, 10 mM imidazol, 10% (v/v) glycerol). After the mixture was disrupted by four passes through a Microfluidizer operated at 30 psi, the cell debris was removed by centrifugation (58,250×g for 30 minutes at 4° C.). Cell lysate containing His₆-tagged proteins was applied at 3 mL/min to a 25 mL Ni-NTA (Qiagen) column in the presence of 10 mM imidazole using a gradiFrac system (Pharmacia). The column was washed with 10 column volumes of the lysis buffer. The bound NS4A₍₂₁₋₃₂₎-GSGS-NS3₍₃₋₁₈₁₎ was eluted with 8 column volumes of buffer A supplemented with 300 mM imidazole. The pooled fractions were further purified by Q-Sepharose column equilibrated in buffer B (50 mM HEPES, pH 7.4, 0.1% (w/v) CHAPS, 10% (v/v) glycerol, 5 mM dithiothreitol (DTT), and 1 M NaCl). The eluant containing NS4A₍₂₁₋₃₂₎-GSGS-NS3₍₃₋₁₈₁₎ was collected. Fractions containing NS4A₍₂₁₋₃₂₎-GSGS-NS3₍₃₋₁₈₁₎ were pooled and further purified by size-exclusion chromatography using the sephacryl-75 column (16×100 cm, Pharmacia) at a flow rate of 0.5 mL/min.

Columns were pre-equilibrated in buffer C (50 HEPES, pH 7.4, 0.1% (w/v) CHAPS, 5 mM DTT, 10% (v/v) glycerol). The purified protein was frozen and stored at −80° C. before use.

### Inhibition Assay Protocol

The HPLC Microbore assay for separation of HCV protease substrate and products was used. The substrate used in the assay was Ac-Asp-Glu-Asp(EDANS)-Glu-Glu-Abu-Ψ-[COOAla]-Ser-Lys(DABCYL)-NH₂ (RET S1, ANASPEC). The buffer used in the assay included 50 mM Tris buffer, pH 7.4, 100 mM NaCl, 20% glycerol, and 0.012% CHAPS.

A solution containing 10 mM DTT, 5 μM substrate RET S1, and 10 μM a test compound in the buffer solution was prepared. The solution (80 μL) was added to each well of a 96-well plate. 20 μL of 10 nM NS3/4A protease in the buffer solution was added to each well to initiate reaction. The resulting assay solution had a total volume of 100 μL. The final concentration of NS3/4A protease was 2 nM, which was lower than the Km of substrate RET S1.

The assay solution was incubated for 30 minutes at 37° C. with 5% CO₂. The reaction was then terminated by addition of 100 μL of 1% TFA. 200 μL aliquot was transferred to each well of Agilent 96-well plates for the next step.

Reaction products were analyzed using reverse phase HPLC described below. The HPLC system included: Agilent 1100; Degasser G1379 A; Binary pump G1312A; Autosampler G1367A; Column thermostated chamber G1316A; Diode array detector G1315B; Column: Agilent, ZORBAX Eclipse XDB-C18; 4.6 mm; 5 μm; P/N 993967-902; Column thermostat: room temperature; Injection volume: 100 μL; Solvent A=HPLC grade water+0.09% TFA; Solvent B=HPLC grade acetonitrile+0.09% TFA. Total HPLC running time was 7.6 minutes with a linear gradient from 25 to 50% solvent B in 4 minutes, 50% solvent B for 30 seconds, and a gradient from 50 to 25% solvent B for additional 30 seconds. The column was re-equilibrated with 25% solvent B for 2.6 minutes before next sample was injected. The IC₅₀ value (the concentration at which 50% inhibition of NS3/4A was observed) was calculated for each test compound based on the HPLC results.

### Results

Compounds 1-48 were tested and all exhibited inhibition of NS3/4A protease activity. 28 compounds exhibited IC₅₀ values of no more than 0.5 μM and 20 compounds exhibited IC₅₀ values in the range of 0.5-5.0 μM.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of formula (I): 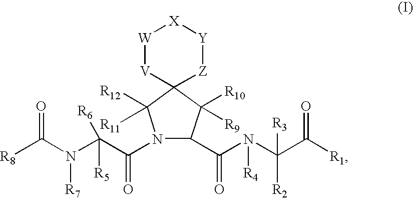
wherein
each of V, W, X, Y, and Z, independently, is O, S, S(O), S(O)₂, C(Rₐ₁Rₐ₂), C(O), N(Rₐ₁), or deleted; or V and W, W and X, X and Y, or Y and Z, together are aryl, C₃-C₂₀ cycloalkyl, or C₁-C₂₀ heterocycloalkyl; provided that at least one of V, W, X, Y, and Z is C(O), at most one of V, W, X, Y, and Z is deleted, and at most two of V, W, X, Y, and Z are O, S, S(O), S(O)₂, C(O), or N(Rₐ₁);
R₁ is H, OR_{b1}, C₁-C₁₀ alkyl, C₂-C₁₀ alkenyl, C2-C10 alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C(O)-N(R_{b1}R_{b2}), N(R_{b1})—C(O)Rb2 , N(R_{b1}R_{b2}), or N(R_{b1})—S(O)₂R_{b2};
each of R₂ and R₃, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or, heteroaryl; or R₂ and R₃, together with the carbon atom to which they are attached, are C₃-C₂₀ cycloalkyl or C₁-C₂₀ heterocycloalkyl;
each of R₅ and R₆, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or, heteroaryl;
R₈ is OR_{c1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, N(R_{c1}R_{c2}), or N(R_{c1})—C(O)—N(R_{c2}R_{c3}); and
each of R₄, R₇, R₉, R₁₀, R₁₁, and R₁₂, independently, is H or C₁-C₁₀ alkyl; in which each of Rₐ₁, Rₐ₂, R_{b1}, R_{b2}, R_{b3}, R_{c1}, R_{c2}, and R_{c3}, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkeny heterocycloalkyl, C₁-C₂₀ heterocycloalkenyl, aryl, or heteroaryl; or Rₐ₁ and Rₐ₂, together with the atom to which they are attached, are C₃-C₂₀ cycloalkyl or C₁-C₂₀ heterocycloalkyl.

2. The compound of claim 1, wherein at least one of V and W, W and X, X and Y, or Y and Z, taken together, is aryl, or C₁-C₂₀ heterocycloalkyl optionally substituted with OR or N(R)—C(O)R′; the remaining V, W, X, Y, and Z, independently, is O, S, C(Rₐ₁Rₐ₂), C(O), N(Rₐ₁), or deleted; in which each of R and R′, independently, is H or C₁-C₁₀ alkyl optionally substituted with aryl or C₂-C₁₀ alkenyl, and each of Rₐ₁ and Rₐ₂, independently, is H, C₂-C₁₀ alkenyl, C₂-C₁₀ alkynyl, or C₁-C₁₀ alkyl optionally substituted with aryl.

3. The compound of claim 2, wherein R₈ is OR_{c1}, N(R_{c1}R_{c2}), N(R_{c1})—C(O)—N(R_{c2}Rc₃), or C₁-C₁₀ alkyl substituted with C₃-C₂₀ cycloalkyl, or N(R_{c1})—C(O)R_{c2}, in which each of R_{c1}, R_{c2}, and R_{c3}, independently, is H, C₁-C₁₀ alkyl, C₃-C₂₀ cycloalkyl, C₁-C₂₀ heterocycloalkyl, aryl, or heteroaryl.

4. The compound of claim 3, wherein R₈ is

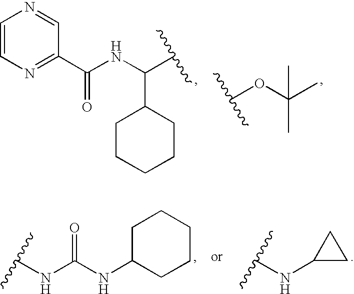

5. The compound of claim 4, wherein R₁ is OR_{b1}, C(O)—N(R_{b1}R_{b2}), or N(R_{b1})—S(O)₂ R_{b2}, in which each of R_{b1} and R_{b2}, independently, is H, C₁-C₁₀ alkyl, C₃-C₂₀ cycloalkyl, C₁-C₂₀ heterocycloalkyl, aryl, or heteroaryl.

6. The compound of claim 5, wherein each of R₂ and R₃, independently, is H or C₁-C₁₀ alkyl; or R₂ and R₃, together with the carbon atom to which they are attached, are C₃-C₂₀ cycloalkyl substituted with C₂-C₁₀ alkenyl.

7. The compound of claim 6, wherein each of R₅ and R₆, independently, is H or isobutyl.

8. The compound of claim 7, wherein the compound is one of compounds 1, 8-11, 13-34, 36, 37, and 40.

9. The compound of claim 1, wherein each of V, W, X, Y, and Z, independently, is O, S, C(Rₐ₁Rₐ₂), C(O), N(Rₐ₁), or deleted; in which each of Rₐ₁ and Rₐ₂, independently, is H or C₁-C₁₀ alkyl.

10. The compound of claim 9, wherein R₈ is OR_{c1}, or C₁-C₁₀ alkyl substituted with C₃-C₂₀ cycloalkyl, N(R_{c1})—C(O)R_{c2}, or N(R_{c1})—C(O)—N(R_{c2}R_{c3}); in which each of R_{c1}, R_{c2}, and R_{c3}, independently, is H, C₁-C₁₀ alkyl, C₃-C₂₀ cycloalkyl, C₁-C₂₀ heterocycloalkyl, aryl, or heteroaryl.

11. The compound of claim 10, wherein R₈ is

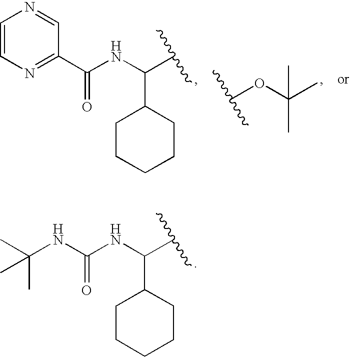

12. The compound of claim 11, wherein R₁ is C(O)—N(R_{b1}R_{b2}), in which each of R_{b1} and R_{b2}, independently, is H, C₁-C₁₀ alkyl, C₃-C₂₀ cycloalkyl, C₁-C₂₀ heterocycloalkyl, aryl, or heteroaryl.

13. The compound of claim 12, wherein each of R₂ and R₃, independently, is H or C₁-C₁₀ optionally substituted with C₃-C₂₀ cycloalkyl.

14. The compound of claim 13, wherein each of R₅ and R₆, independently, is H or isobutyl.

15. The compound of claim 7, wherein the compound is one of compounds 2-7, 12, 35, 38, and 39.

16. The compound of claim 1, wherein R₈ is 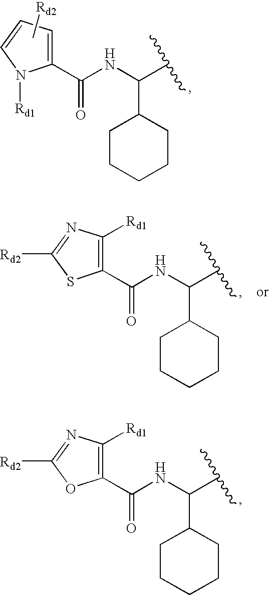
in which each of R_{d1} and R_{d2}, independently, is H or C₁-C₁₀ alkyl.

17. The compound of claim 16, wherein R₁ is OR_{b1}, C(O)—N(R_{b1}R_{b2}), or N(R_{b1})—S(O)₂R_{b2}, in which each of R_{b1} and R_{b2}, independently, is H, C₁-C₁₀ alkyl, C₃-C₂₀ cycloalkyl, C₁-C₂₀ heterocycloalkyl, aryl, or heteroaryl.

18. The compound of claim 17, wherein each of R₂ and R₃, independently, is H or C₁-C₁₀ alkyl; or R₂ and R₃, together with the carbon atom to which they are attached, are C₃-C₂₀ cycloalkyl substituted with C₂-C₁₀ alkenyl.

19. The compound of claim 18, wherein each of R₅ and R₆, independently, is H or isobutyl.

20. The compound of claim 1, wherein the compound is one of compounds 41-48.

21. A method for treating hepatitis C virus infection, comprising administering to a subject in need thereof an effective amount of a compound of formula (I): 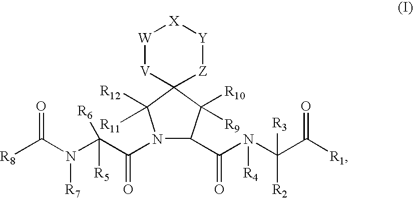
wherein
each of V, W, X, Y, and Z, independently, is O, S, S(O), S(O)₂, C(Rₐ₁Rₐ₂), C(O), N(Rₐ₁), or deleted; or V and W, W and X, X and Y, or Y and Z, together are aryl, C₃-C₂₀ cycloalkyl, or C₁-C₂₀ heterocycloalkyl; provided that at least one of V, W, X, Y, and Z is C(O), at most one of V, W, X, Y, and Z is deleted, and at most two of V, W, X, Y, and Z are O, S, S(O), S(O)₂, C(O), or N(Rₐ₁);
R₁ is H, OR_{b1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, C(O)—N(Rb₁R_{b2}), N(R_{b1})—C(O)R_{b2}, N(R_{b1}R_{b2}), or N(R_{b1})—S(O)₂R_{b2};
each of R₂ and R₃, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or, heteroaryl; or R₂ and R₃, together with the carbon atom to which they are attached, are C₃-C₂₀ cycloalkyl or C₁-C₂₀ heterocycloalkyl;
each of R₅ and R₆, independently, is H, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, or, heteroaryl;
R₈ is OR_{c1}, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, aryl, heteroaryl, N(R_{c1}R_{c2}), or N(R_{c1})—C(O)—N(R_{c2}R_{c3}); and
each of R₄, R₇, R₉, R₁₀, R₁₁, and R₁₂, independently, is H or C₁-C₁₀ alkyl; in which each of Rₐ₁, Rₐ₂, R_{b1}, R_{b2}, R_{b3}, R_{c1}, R_{c2}, and R_{c3}, independently, is H, halo, C₁-C₁₀ alkyl, C₂-C₁₀ alkenyl, C₂-C₁₀ alkynyl, C₃-C₂₀ cycloalkyl, C₃-C₂₀ cycloalkeny heterocycloalkyl, C₁-C₂₀ heterocycloalkenyl, aryl, or heteroaryl; or Rₐ₁ and Rₐ₂, together with the atom to which they are attached, are C₃-C₂₀ cycloalkyl or C₁-C₂₀ heterocycloalkyl.

22. The method of claim 21, wherein the compound is one of compounds 1-48.

23. A pharmaceutical composition, comprising the compound of claim 1 and a pharmaceutically acceptable carrier.

24. The composition of claim 23, further comprising a second antiviral agent.

25. The composition of claim 23, wherein the compound is one of compounds 1-48.
